(19) United States
(12) Patent Application Publication
(10) Pub. No.: US 2005/0197518 A1
(43) Pub. Date: Sep. 8, 2005

(54) Method of converting C9 aromatics-comprising mixtures to xylene isomers

(76) Inventors: Jeffrey T. Miller, Naperville, IL (US); George A. Huff, Naperville, IL (US); Brian J. Henley, Sandwich, IL (US)

Correspondence Address: BP America Inc., Docket Clerk, BP Legal, M.C. 5East, 4101 Winfield Road, Warrenville, IL, 60555, US

(21) Appl. No.: 10/794,932

(22) Filed: Mar. 4, 2004

(51) Int. Cl. C07C 15/12

(52) U.S. Cl. 585/475

(57) ABSTRACT

Disclosed herein is a method of making xylene isomers. More specifically, the method includes contacting a C₉ aromatics-comprising feed with a catalyst under conditions suitable for converting the feed to an intermediate product stream comprising xylene isomers, separating at least a portion of the xylene isomers from the intermediate product stream, and recycling to the feed the xylene isomers-lean intermediate product stream. Alternatively, the method of making xylene isomers includes contacting a feed comprising C₉ aromatics and less than about 30 wt. % benzene, based on the total weight of the feed, with a non-sulfided, large-pore zeolite impregnated with a Group VIB metal oxide, under conditions suitable for converting the feed to a product stream comprising xylene isomers. The disclosed method is characterized by unexpectedly high ratios of xylene isomers to ethylbenzene, xylene isomers to C₉ aromatics (e.g., methylethylbenzene), xylene isomers to C₁₀ aromatics, trimethylbenzene to methylethylbenzene, benzene to ethylbenzene, in the product of the conversion, and the high conversion of C₉ aromatics and methylethylbenzene.

### BACKGROUND OF THE INVENTION

1. Field of the Invention

The invention generally relates to a method of catalytically converting aromatic hydrocarbons and, more specifically, to a method of disproportionating and transalkylating benzene, toluene, and C₉ aromatics to xylene isomers.

2. Brief Description of Related Technology

Hydrocarbon mixtures containing C₈ aromatics are often products of oil refinery processes including, but not limited to, catalytic reforming processes. These reformed hydrocarbon mixtures typically contain C₆₋₁₁ aromatics and paraffins, most of the aromatics of which are C₇₋₉ aromatics. These aromatics can be fractionated into their major groups, i.e., C₆, C₇, C₈, C₉, C₁₀, and C₁₁ aromatics. Present in the C₈ aromatics fraction are non-aromatics, which comprise about 10 weight percent (wt. %) to about 30 wt. % based on the total weight of the C₈ fraction. The balance of this fraction is comprised of C₈ aromatics. Most commonly present among the C₈ aromatics are ethylbenzene (“EB”), and xylene isomers, including meta-xylene (“mX”), ortho-xylene (“oX”), and para-xylene (“pX”). Together, the xylene isomers and ethylbenzene are collectively referred to in the art and herein as “C₈ aromatics.” Typically, when present among the C₈ aromatics, ethylbenzene is present in a concentration of about 15 wt. % to about 20 wt. %, based on the total weight of the C₈ aromatics, with the balance (e.g., up to about 100 wt. %) being a mixture of xylene isomers. The three xylene isomers typically comprise the remainder of the C₈ aromatics, and are generally present at an equilibrium weight ratio of about 1:2:1 (oX:mX:pX). Thus, as used herein, the term “equilibrated mixture of xylene isomers” refers to a mixture containing the isomers in the weight ratio of about 1:2:1 (oX:mX:pX).

The product (or reformate) of a catalytic reforming process contains C₆₋₈ aromatics (i.e., benzene, toluene, and C₈ aromatics, which are collectively referred to as “BTX”). Byproducts of the process include hydrogen, light gas, paraffins, naphthenes, and heavy C₉₊ aromatics. The BTX present in the reformate (especially toluene, ethylbenzene, and xylene) are known to be useful gasoline additives. However, due to environmental and health concerns, the presence of certain aromatics (especially benzene) in gasoline has been greatly reduced and disfavored. Nonetheless, the constituent parts of BTX can be separated in downstream unit operations for use in other capacities. Alternatively, benzene can be separated from the BTX and the resulting mixture of toluene and C₈ aromatics can be used as additives to boost the octane rating of gasoline, for example.

Benzene and xylenes (especially para-xylene) are more highly valued than toluene due to their usefulness in making other products. For example, benzene can be used to make styrene, cumene, and cyclohexane. Benzene also is useful in the manufacture of rubbers, lubricants, dyes, detergents, drugs, and pesticides. Among the C₈ aromatics, ethylbenzene generally is useful in making styrene when such ethylbenzene is a reaction product of ethylene and benzene. However, due to purity problems, the ethylbenzene that is created as a result of transalkylation and/or disproportionation cannot be used for styrene production. Meta-xylene is useful in making isophthalic acid, which itself is useful to make specialty polyester fibers, paints, and resins. Ortho-xylene is useful in making phthalic anhydride, which itself is useful to make phthalate-based plasticizers. Para-xylene is a raw material useful in making terephthalic acids and esters, which are used to make polymers, such as poly(butene terephthalate), poly(ethylene terephthalate), and poly(propylene terephthalate). While ethylbenzene, meta-xylene, and ortho-xylene are useful raw materials, demands for these chemicals and materials made therefrom are not as great as the demand for para-xylene and the materials made from para-xylene.

In view of the higher values placed on benzene, CB aromatics, and products made therefrom, processes have been developed to dealkylate toluene to benzene, disproportionate toluene to benzene and C₈ aromatics, and transalkylate toluene and C₉₊ containing aromatics to C₈ aromatics. These processes are generally described in Kirk Othmer's “Encyclopedia of Chemical Technology,” 4^{th} Ed., Supplement Volume, pp. 831-863 (John Wiley & Sons, New York, 1995), the disclosure of which is incorporated herein by reference.

Specifically, toluene disproportionation (“TDP”) is a catalytic process wherein two moles of toluene are converted to one mole of xylene and one mole of benzene, such as:

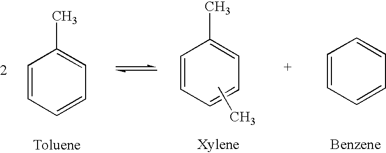

Other disproportionation reactions include a catalytic process wherein two moles of a C₉ aromatic are converted to one mole of toluene and heavier hydrocarbon components (i.e., C₁₀₊ heavies), such as:

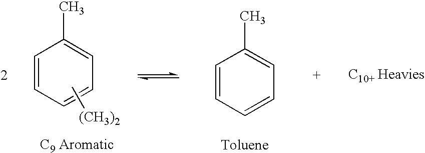

Toluene transalkylation is a reaction between one mole of toluene and one mole of C₉ aromatic (or higher aromatic) to produce two moles of xylene, such as:

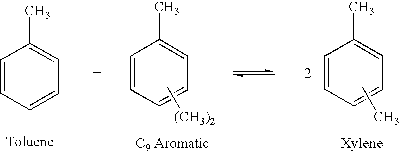

Other transalkylation reactions involving C₉ aromatics (or higher aromatics) include the reaction with benzene to produce toluene and xylene, such as:

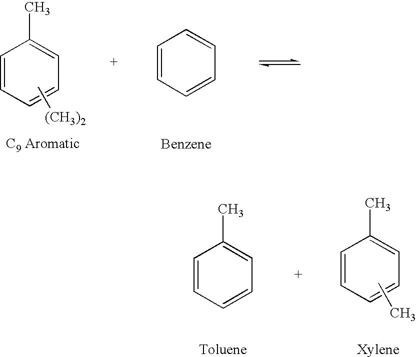

As shown in the foregoing reactions, the methyl and ethyl groups associated with the C₉ aromatic and xylene molecules are shown generically as such groups can be found bound to any available ring-forming carbon atoms to form the various isomeric configurations of the molecule. Mixtures of xylene isomers can be further separated into their constituent isomers in downstream processes. Once separated, the isomers can be further processed (e.g., isomerized) and recycled to obtain a substantially pure para-xylene, for example.

In theory and in view of the foregoing reactions, a mixture comprising C₉ aromatics can be converted to xylenes and/or benzene. Mixtures of xylenes and benzene can be separated from one another by fractional distillation, for example. Heretofore, however, it has not been known how the reactions can be carried out in a manner such that a pure xylenes product is obtainable from a given feed comprising C₉ aromatics.

U.S. Pat. Nos. 5,907,074; 5,866,741; 5,866,742; and, 5,804,059, each assigned to the Phillips Petroleum Company (“Phillips”), generally disclose disproportionation and transalkylation reactions wherein certain fluid feeds containing C₉₊ aromatics are converted to BTX. Though these patents state that the origin of the fluid feeds is not critical, each expresses a strong preference for fluid feeds derived from the heavies fraction of a product obtained by a hydrocarbon (particularly gasoline) aromatization reaction, which typically is carried out in a fluid catalytic cracking (“FCC”) unit. Low-value, liquid feeds comprising large (or long) hydrocarbons are vaporized in the FCC unit and, in the presence of a suitable catalyst, are cracked into lighter molecules capable of forming products that can be blended into higher-valued diesel fuel and high-octane gasoline. Byproducts of the FCC unit include a lower-valued, liquid heavies fraction, which constitutes the fluid feeds preferred according to the teachings of these patents. The very origin of the preferred fluid feeds, suggests that the feeds comprise sulfur-comprising compounds, paraffins, olefins, naphthenes, and polycyclic aromatics (“polyaromatics”).

According to the '074 patent, BTX are generally substantially absent from the feeds preferred therein and, therefore, no significant transalkylation of BTX occurs as a side reaction to the primary disproportionation and transalkylation reactions. The primary reactions described therein occur in the presence of a hydrogen-containing fluid and a catalyst comprising a metal oxide-promoted, Y-type zeolite having incorporated therein an activity modifier (i.e., oxides of sulfur, silicon, phosphorus, boron, magnesium, tin, titanium, zirconium, germanium, indium, lanthanum, cesium, and combinations of two or more thereof. The activity modifier helps to combat the deactivating effect (or poisoning effect) that sulfur-comprising compounds have on metal oxide impregnated catalysts.

According to the '741, '742, and '059 patents, BTX are generally substantially absent from the feeds preferred therein and, therefore, no significant transalkylation of BTX occurs as a side reaction to the primary disproportionation and transalkylation reactions. However, BTX can be present where alkylation of such chemicals by the C₉₊ aromatics is secondarily desired. According to the '741 patent, these primary and secondary reactions occur in the presence of a hydrogen-containing fluid and a catalyst comprising a beta-type zeolite having incorporated therein an activity promoter (e.g., molybdenum, lanthanum, and oxides thereof). According to the '742 patent, the primary and secondary reactions occur in the presence of a hydrogen-containing fluid and a catalyst comprising a beta-type zeolite having incorporated therein a metal carbide. According to the '059 patent, the primary and secondary reactions occur in the presence of a hydrogen-containing fluid and a catalyst comprising a metal oxide-promoted, mordenite-type zeolite.

The stated purpose underlying the teachings of each of the foregoing patents is to convert C₉₊ aromatics to BTX. Given this purpose, the patents disclose a specific combination of fluid feeds, catalysts, and reaction conditions suitable to obtain BTX. These patents do not, however, disclose or teach how to obtain any single BTX component (much less xylene isomers) to the exclusion of the other BTX components. With respect to each of these, the presence of sulfur in the fluid feeds detrimentally converts the metal or metal oxide in the catalyst to a metal sulfide over time. Metal sulfides have a much lower hydrogenation activity than metal oxides and, therefore, the sulfur poisons the activity of the catalyst. Furthermore, the olefins, paraffins, and polyaromatics present in the feed rapidly deactivate the catalyst, and are converted to undesirable light gas.

In contrast to the foregoing patents, U.S. Patent Application Publication No. 2003/0181774 A1 (Kong et al.) discloses a transalkylation method of catalytically converting benzene and C₉₊ aromatics to toluene and C₈ aromatics. According to Kong et al., the method should be carried out in the presence of hydrogen in a gas-solid phase, fixed-bed reactor having a transalkylation catalyst comprising H-zeolite and molybdenum. The stated purpose behind Kong et al.'s method is to maximize production of toluene for subsequent use as a feed in a downstream selective disproportionation reactor, and to use the obtained C₈ aromatics by-product as a feed in a downstream isomerization reactor. By selective disproportionation of the toluene to para-xylene, Kong et al. suggest how to ultimately convert a mixture of benzene and C₉₊ aromatics to para-xylene. However, such a suggestion disadvantageously requires multiple reaction vessels (e.g., a transalkylation reactor, and a disproportionation reactor) and, importantly, does not teach how to maximize the amount of xylene isomers produced from the transalkylation reaction, while concomitantly minimizing the production of toluene and ethylbenzene.

U.S. Patent Application Publication No. 2003/0130549 A1 (Xie et al.) discloses a method of selectively disproportionating toluene to obtain benzene and a xylene isomers stream rich in para-xylene, and transalkylating a mixture of toluene and C₉₊ aromatics to obtain benzene and xylene isomers. According to Xie et al., the different reactions are carried out in the presence of hydrogen in separate reactors each containing a suitable catalyst (i.e., a ZSM-5 catalyst for the selective disproportionation and a mordenite, MCM-22 or beta-zeolite for the transalkylation). Downstream processing is used to obtain para-xylene from the produced xylene isomers. The method disclosed by Xie et al. suggests that large volumes of benzene and ethylbenzene are desirably produced. Xie et al., however, do not suggest how to maximize the amount of xylene isomers produced from the transalkylation reaction, while concurrently minimizing the production of benzene and ethylbenzene.

U.S. Patent Application Publication No. 2001/0014645 A1 (Ishikawa et al.) discloses a method of disproportionating C₉₊ aromatics into toluene and transalkylating C₉₊ aromatics and benzene to toluene and C₈ aromatics for use as gasoline additives. The use of benzene as a reactant in the transalkylation reaction suggests an attempt by Ishikawa et al. to rid low-value gasoline fractions of benzene. Given the stated use and suggestion to rid gasoline of benzene, one skilled in the art would desire ethylbenzene in the C₈ aromatics to maximize gasoline yields. Moreover, the skilled artisan will take precautions to ensure that the produced ethylbenzene is not unintentionally cracked to a benzene—which is sought to be removed from gasoline fractions. The disclosed reactions are carried out in the presence of hydrogen and a large-pore zeolite impregnated with a Group VIB metal and preferably sulfided. Generally, portions of the benzene and C₉₊ aromatics are converted to a product stream mostly comprising BTX. From the BTX product stream, benzene is removed and recycled back to the feed. Ultimately, toluene and C₈ aromatics are obtained from the benzene/C₉₊ aromatics feed. The transalkylating reaction is carried out with a large molar excess of benzene to C₉₊ aromatics (i.e., between 5:1 to 20:1) to obtain toluene and C₈ aromatics (including ethylbenzene). Ishikawa et al., however, do not suggest how to maximize the amount of xylene isomers produced in the transalkylation reaction, while also minimizing the production of toluene, benzenes, and C₁₀ aromatics.

Generally, the prior art does not sufficiently teach or suggest to one of ordinary skill in the art how to obtain xylene isomers from a mixture that contains C₉ aromatics and, optionally, toluene and benzene.

### SUMMARY OF THE INVENTION

Disclosed herein is a method of making xylene isomers. More specifically, the method includes contacting a C₉ aromatics-comprising feed with a catalyst under conditions suitable for converting the feed to an intermediate product stream comprising xylene isomers, separating at least a portion of the xylene isomers from the intermediate product stream, and recycling to the feed the xylene isomers-lean intermediate product stream.

In one embodiment, the method of making xylene isomers includes contacting a feed comprising C₉ aromatics and less than about 30 wt. % benzene, based on the total weight of the feed, with a non-sulfided, large-pore zeolite impregnated with a Group VIB metal oxide, under conditions suitable for converting the feed to a product stream comprising xylene isomers.

In another embodiment, a method of converting a C₉ aromatics-comprising feed to a product stream containing xylene isomers includes contacting the feed with a catalyst under conditions suitable to yield a weight ratio of xylene isomers to ethylbenzene in the product stream of at least about 6 to 1.

In a further embodiment, the method of converting a C₉ aromatics-comprising feed to a product stream containing xylene isomers includes contacting the feed with a catalyst under conditions suitable to yield a weight ratio of xylene isomers to methylethylbenzene in the product stream of at least about 1 to 1.

In another embodiment, the method of converting a C₉ aromatics-comprising feed to a product stream containing xylene isomers includes contacting the feed with a catalyst under conditions suitable to yield a weight ratio of xylene isomers to C₁₀ aromatics in the product stream of at least about 3 to 1.

In a yet another embodiment, the method of converting a C₉ aromatics-comprising feed to a product stream containing xylene isomers includes contacting the feed with a catalyst under conditions suitable to yield a weight ratio of trimethylbenzene to methylethylbenzene in the product stream of at least about 1.5 to 1.

In a still further embodiment, the method of converting a C₉ aromatics-comprising feed to a product stream containing xylene isomers includes contacting the feed with a catalyst under conditions suitable to yield a weight ratio of benzene to ethylbenzene in the product stream of at least about 2 to 1.

In a further embodiment, the method of converting a C₉ aromatics-comprising feed to a product stream containing xylene isomers includes contacting the feed with a catalyst under conditions suitable to yield a weight ratio of C₉ aromatics present in the feed to that present in the product stream is at least about 4 to 1.

In still a further embodiment, the method of converting a C₉ aromatics-comprising feed to a product stream containing xylene isomers includes contacting the feed with a catalyst under conditions suitable to yield a weight ratio of methylethylbenzene in the feed to that present in the product stream of at least about 2 to 1.

Additional features of the invention may become apparent to those skilled in the art from a review of the following detailed description, taken in conjunction with the drawing, the examples, and the appended claims.

### BRIEF DESCRIPTION OF THE DRAWINGS

For a more complete understanding of the invention, reference should be made to the following detailed description and accompanying drawings, wherein:

FIG. 1 is a schematic generally illustrating the apparatus that can be used to carry out the disclosed methods;

FIG. 2 is a schematic generally illustrating the process flow of a steady state conversion of C₉ aromatics using a mordenite catalyst; and,

FIG. 3 is a schematic generally illustrating the process flow of a steady state conversion of C₉ aromatics using a molybdenum-impregnated mordenite catalyst.

While the disclosed method is susceptible of embodiments in various forms, there are illustrated in the drawings (and will hereafter be described) specific embodiments of the invention, with the understanding that the disclosure is intended to be illustrative, and is not intended to limit the invention to the specific embodiments described and illustrated herein.

### DETAILED DESCRIPTION OF THE INVENTION

The invention generally relates to a method of making xylene isomers, which are especially suitable as a chemical feedstock for the production of para-xylene. More specifically, the method includes contacting a C₉ aromatics-comprising feed with a catalyst under conditions suitable for converting the feed to an intermediate product stream comprising xylene isomers, separating at least a portion of the xylene isomers from the intermediate product stream, and recycling to the feed the xylene isomers-lean intermediate product stream. Alternatively, the method of making xylene isomers includes contacting a feed comprising C₉ aromatics and less than about 30 wt. % benzene, based on the total weight of the feed, with a non-sulfided, large-pore zeolite impregnated with a Group VIB metal oxide, under conditions suitable for converting the feed to a product stream comprising xylene isomers.

Suitable feeds for use in accordance with the disclosed inventive methods include those ultimately obtained from crude oil refining processes. Generally, crude oil is desalted and thereafter distilled into various components. The desalting step generally removes metals and suspended solids that could cause catalyst deactivation in downstream processes. The product obtained from the desalting step subsequently undergoes atmospheric or vacuum distillation. Among the fractions obtained via atmospheric distillation are crude or virgin naphtha, gasoline, kerosene, light fuel oil, diesel oils, gas oils, lube distillates, and heavy bottoms, which often are further distilled via vacuum distillation methods. Many of these fractions can be sold as finished products or can be further processed in downstream unit operations capable of changing the molecular structure of the hydrocarbon molecules either by breaking them into smaller molecules, combining them to form a larger more highly-valued molecule, or reshaping them into more highly-valued molecules. For example, crude or virgin naphtha obtained from the distillation step can be passed with hydrogen through a hydrotreating unit, which converts olefins to paraffins, and removes impurities such as sulfur, nitrogen, oxygen, halides, heteroatoms, and metal impurities that can deactivate downstream catalysts. Exiting the hydrotreating unit is a treated gas lean or substantially free of impurities, a hydrogen-rich gas, and streams containing hydrogen sulfide and ammonia. The light hydrocarbons are sent to a downstream unit operation (a “reformer”) to convert those hydrocarbons (e.g., nonaromatics) into hydrocarbons having better gasoline properties (e.g., aromatics). The treated gas, generally containing aromatics (typically in the boiling range of C₆₋₁₀ aromatics), can serve as a feed suitable for conversion in accordance with the disclosed inventive methods.

Alternatively, a hydrocracking unit can take a feed similar to the one sent to a FCC unit and converts that feed to light hydrocarbons having poor gasoline properties (i.e., naphtha) and little to no sulfur or olefins. The light hydrocarbons are then sent to a reformer to convert those hydrocarbons into hydrocarbons having better gasoline properties (e.g., aromatics). Exiting the reformer is a reformate that includes not only aromatics (typically in the boiling range of C₆₋₁₀ aromatics) but also paraffins. The reformate is substantially free of sulfur and olefins, but includes paraffins and polyaromatics. Thus, in a subsequent step, paraffins and polyaromatics are removed to yield a product stream containing C₉ aromatics. Such a product stream can serve as a feed suitable for conversion in accordance with the disclosed inventive methods.

The composition of crude oil can vary significantly depending upon its source. Moreover, feeds suitable for use in accordance with the inventive methods disclosed herein are typically obtained as products of a variety of upstream unit operations and, of course, can vary depending upon the reactants/materials supplied to those unit operations. Oftentimes, the origin of those reactants/materials will dictate the composition of the feed obtained as a product of the unit operations.

The C₉ aromatics-comprising feed generally includes C₉ aromatics. As used herein, the term “aromatic” defines a major group of unsaturated cyclic hydrocarbons containing one or more rings, typified by benzene, which has a six-carbon ring containing three double bonds. See generally, “Hawley's Condensed Chemical Dictionary,” at p. 92 (13^{th} Ed., 1997). As used herein, the term “C₉ aromatics” means a mixture that includes any aromatic compound having nine carbon atoms. Preferably, the C₉ aromatics include 1,2,4-trimethylbenzene (psuedocumene), 1,2,3-trimethylbenzene (hemimellitene), 1,3,5-trimethylbenzene (mesitylene), meta-methylethylbenzene, ortho-methylethylbenzene, para-methylethylbenzene, iso-propylbenzene, and n-propylbenzene.

Along with the C₉ aromatics, the feed typically will include numerous other hydrocarbons, many of which are only present in trace amounts. For example, the feed should be substantially free of paraffins and olefins. A feed that is substantially free of paraffins and olefins preferably comprises less than about 3 wt. % of each of paraffins and olefins, and more preferably less than about 1 wt. % of each of paraffins and olefins, based on the total weight of the feed. Furthermore, the feed should be substantially free of sulfur (e.g., elemental sulfur and sulfur-containing hydrocarbons and non-hyrdocarbons). A feed that is substantially free of sulfur preferably comprises less than about 1 wt. % sulfur, more preferably less than about 0.1 wt. % sulfur, and even more preferably less than about 0.01 wt. % sulfur, based on the total weight of the feed.

In various preferred embodiments, the feed is substantially free of xylene isomers, toluene, ethylbenzene, and/or benzene. A feed that is substantially free of xylene isomers preferably comprises less than about 3 wt. % xylene isomers, and more preferably less than about 1 wt. % xylene isomers, based on the total weight of the feed. A feed that is substantially free of toluene preferably comprises less than about 5 wt. % toluene, and more preferably less than about 3 wt. % toluene, based on the total weight of the feed. A feed that is substantially free ethylbenzene preferably comprises less than about 5 wt. % of ethylbenzene, and more preferably less than about 3 wt. % ethylbenzene, based on the total weight of the feed. A feed that is substantially free of benzene preferably comprises less than about 5 wt. % benzene, and more preferably less than about 3 wt. % benzene, based on the total weight of the feed.

In certain embodiments, however, the feed can include significant amounts of one or both of toluene and benzene. For example, in certain embodiments, the feed can include up to about 50 wt. % toluene, based on the total weight of the feed. Preferably, however, the feed includes less than about 50 wt. % toluene, more preferably less than about 40 wt. % toluene, even more preferably less than about 30 wt. % toluene, and most preferably less than about 20 wt. % toluene, based on the total weight of the feed. Similarly, in certain embodiments, the feed can include up to about 30 wt. % benzene, based on the total weight of the feed. Preferably, however, the feed includes less than about 30 wt. % benzene, and more preferably, less than about 20 wt. % benzene, based on the total weight of the feed.

Still further, in various embodiments, the feed can be substantially free of C₁₀₊ aromatics. The feed, however, need not be substantially free of C₁₀₊ aromatics. Generally, C₁₀₊ aromatics (“A₁₀₊”) will include benzenes having one or more hydrocarbon functional groups which, in the aggregate, have four or more carbons. Examples of such C₁₀₊ aromatics include, but are not limited to, C₁₀ aromatics (“A₁₀”), such as butylbenzene, (including isobutylbenzene and tertiarybutylbenzene), diethylbenzene, methylpropylbenzene, dimethylethylbenzene, tetramethylbenzene, and C₁₁ aromatics, such as trimethylethylbenzene, and ethylpropylbenzene, for example. Examples of C₁₀₊ aromatics also can include naphthalene, and methyinaphthalene. A feed that is substantially free of C₁₀₊ aromatics preferably comprises less than about 5 wt. % C₁₀₊ aromatics, and more preferably less than about 3 wt. % C₁₀₊ aromatics, based on the total weight of the feed.

As used herein the term “C₈ aromatics” means a mixture containing predominantly xylene isomers and ethylbenzene. In contrast, the term “xylene isomers,” as used herein, means a mixture containing meta-, ortho-, and para-xylenes, wherein the mixture is substantially free of ethylbenzene. Preferably, such a mixture contains less than three weight percent ethylbenzene based on the combined weight of the xylene isomers and any ethylbenzene. More preferably, however, such a mixture contains less than about one weight percent ethylbenzene.

As noted above, in some embodiments of the inventive method, the feed is catalytically converted to an intermediate product stream comprising xylene isomers, at least a portion of the xylene isomers is separated from the intermediate product stream, and the intermediate product stream is thereafter recycled to the feed. In a first pass, the product of the conversion is referred to as an “intermediate product stream” and, once at least a portion of the xylene isomers are removed therefrom, the stream is recycled. In other embodiments, however, the “intermediate product stream,” can be considered as the “product stream” as it contains xylene isomers, which are the particular aromatics sought after in the conversion. Accordingly, in these embodiments, the method can be described as one in which the feed is catalytically converted to a product stream comprising xylene isomers, the xylene isomers are separated from the product stream, and the product stream is thereafter recycled to the feed. In these embodiments, the recycled stream, whether referred to as an “intermediate product stream” or a “product stream,” preferably contains no (or only trace amounts on xylene isomers and contains predominantly unreacted feed, toluene, and/or benzene.

In a further embodiment of the inventive method, the product or intermediate product stream contains xylene isomers and ethylbenzene present in a weight ratio of at least about 6 to 1, preferably at least about 10 to 1, and more preferably at least about 25 to 1. Stated another way, the method of converting a C₉ aromatics-comprising feed to a product stream containing xylene isomers includes contacting the feed with a catalyst under conditions suitable to yield a weight ratio of xylene isomers to ethylbenzene in the product stream of at least about 6 to 1, preferably at least about 10 to 1, and more preferably at least about 25 to 1. Such a high weight ratio xylene isomers to ethylbenzene in the product stream is beneficial in downstream processing where the product stream is to be fractionated into its major constituents, i.e., into aromatics containing 6, 7, 8, and 9 carbons. Typically, further processing of a C₈ aromatics fraction would necessarily involve energy-consuming processing of the ethylbenzene. However, given the substantial absence of ethylbenzene in the liquid reaction product, and the accordingly substantial absence of ethylbenzene in the C₈ aromatics fraction, no such energy-consuming processing is required to rid the fraction of ethylbenzene.

Moreover, the substantial absence of ethylbenzene is particularly desired. As previously noted, though ethylbenzene can be Used as a raw material to make styrene, such ethylbenzene must be in a highly purified form. The particular ethylbenzene that results from disproportionating and transalkylating benzene, toluene, and C₉ aromatics is necessarily present in a mixture containing other aromatics. Separating ethylbenzene from such a mixture is very difficult and very expensive. Consequently, from a practical standpoint this ethylbenzene cannot be used in the manufacture of styrene. In practice, the ethylbenzene would either be used as a gasoline additive (as an octane booster therein) or likely be subjected to further disproportionation to yield light gas (e.g., ethane) and benzene. According to the invention, however, the substantial absence of ethylbenzene in the liquid reaction product and C₈ aromatics fraction would obviate such processing.

In another embodiment of the inventive method, the product or intermediate product stream contains xylene isomers to methylethylbenzene (MEB) in a weight ratio of at least about 1 to 1, preferably at least about 5 to 1, and more preferably at least about 10 to 1. Stated another way, the method of converting a C₉ aromatics-comprising feed to a product stream containing xylene isomers includes contacting the feed with a catalyst under conditions suitable to yield a weight ratio of xylene isomers to methylethylbenzene in the product stream of at least about 1 to 1, preferably at least about 5 to 1, and more preferably at least about 10 to 1. The lack of (or low amounts of) methylethylbenzene in the product and/or intermediate product stream is advantageous in that the there are lower amounts of such unreacted or produced C₉ aromatics that need to be recycled back to the feed for conversion, thus, conserving energy and reducing capital costs.

In yet another embodiment of the inventive method, the product or intermediate product stream contains xylene isomers to C₁₀ aromatics in a weight ratio of at least about 3 to 1, preferably at least about 5 to 1, and more preferably at least about 10 to 1. Stated another way, the method of converting a C₉ aromatics-comprising feed to a product stream containing xylene isomers includes contacting the feed with a catalyst under conditions suitable to yield a weight ratio of xylene isomers to C₁₀ aromatics in the product stream of at least about 3 to 1, preferably at least about 5 to 1, and more preferably at least about 10 to 1. Such high ratios are evidence that the dominant reaction involving the C₉ aromatics is a disproportionation reaction yielding xylene isomers and not a reaction yielding C₁₀ aromatics, toluene, and benzene. The lack of or low amounts of C₁₀ aromatics in the product and/or intermediate product stream is advantageous in that the there are lower amounts of such unreacted or produced C₁₀ aromatics that need to be recycled back to the feed for conversion, thus, conserving energy and reducing capital costs. To the extent that C₁₀ aromatics are present in the intermediate or product stream, such C₁₀ aromatics are predominantly tetramethylbenzene, which can be recycled and more amenable to conversion to xylene isomers. Advantageously, the C₁₀ aromatics do not include much ethyldimethylbenzene and/or diethylbenzene, both of which are more difficult to convert to xylene isomers and, therefore, less likely to be recycled.

In a further embodiment of the inventive method, the product or intermediate product stream contains trimethylbenzene to methylethylbenzene in a weight ratio of at least about 1.5 to 1, preferably at least about 5 to 1, more preferably at least about 10 to 1, and even more preferably at least about 15 to 1. Stated another way, the method includes converting a C₉ aromatics-comprising feed to a product stream containing xylene isomers includes contacting the feed with a catalyst under conditions suitable to yield a weight ratio of trimethylbenzene to methylethylbenzene in the product stream of at least about 1.5 to 1, preferably at least about 5 to 1, more preferably at least about 10 to 1, and even more preferably at least about 15 to 1. To obtain a xylene isomer from trimethylbenzene a single methyl group must be removed from the trimethylbenzene molecule. In contrast, to obtain a xylene isomer from methylethylbenzene, one must substitute a methyl group for the ethyl group on the benzene ring. Such a substitution is difficult to carry out. Consequently high ratios of trimethylbenzene to methylethylbenzene are advantageous in that trimethylbenzene is more amenable to conversion to xylene isomers than is methylethylbenzene and, consequently, is more amenable to recycle.

In a still further embodiment of the inventive method, the product or intermediate product stream contains benzene to ethylbenzene in a weight ratio of at least about 2 to 1, preferably at least about 5 to 1, and more preferably at least about 10 to 1. Stated another way, the method of converting a C₉ aromatics-comprising feed to a product stream containing xylene isomers includes contacting the feed with a catalyst under conditions suitable to yield a weight ratio of benzene to ethylbenzene in the product stream of at least about 2 to 1, preferably at least about 5 to 1, and more preferably at least about 10 to 1. Such high ratios are beneficial given that ethylbenzene of the type obtained during disproportionation and transalkylation reactions involving C₉ aromatics have lower value as a chemical feedstock given the difficulties in separating ethylbenzene from a mixture of other C₈ aromatics. As noted above, a molecule of a C₉ aromatic and benzene can be transalkylated to a molecule of xylene and toluene. Thus, the high ratio of benzene relative to ethylbenzene in the stream can prove useful when considering that portions of the stream can be recycled to increase the yield of xylene isomers.

In another embodiment of the inventive method, the product or intermediate product stream contains C₉ aromatics present in an amount (weight ratio) relative to the amount present in the feed of at least about 4 to 1, preferably at least about 8 to 1, and more preferably at least about 10 to 1. Stated another way, the method of converting a C₉ aromatics-comprising feed to a product stream containing xylene isomers includes contacting the feed with a catalyst under conditions suitable to yield a weight ratio of C₉ aromatics present in the feed to that present in the product stream is at least about 4 to 1, preferably at least about 8 to 1, and more preferably at least about 10 to 1. Such a high conversion is beneficial in that there are lower amounts of unreacted C₉ aromatics that need to be recycled back to the feed for conversion, thus, conserving energy and reducing capital costs.

In yet another embodiment of the inventive method, the feed contains methylethylbenzene present in an amount (weight ratio) relative to the amount present in the product or intermediate product stream of at least about 2 to 1, preferably at least about 10 to 1, and more preferably at least about 20 to 1. Stated another way, the method of converting a C₉ aromatics-comprising feed to a product stream containing xylene isomers includes contacting the feed with a catalyst under conditions suitable to yield a weight ratio of methylethylbenzene present in the feed to that present in the product stream of at least about 2 to 1, preferably at least about 10 to 1, and more preferably at least about 20 to 1. Such a high ratio is evidence that the inventive method effectively converts a high proportion of the methylethylbenzene present among the C₉ aromatics in the feed. Indeed, the high ratios show that the reactions are effective to convert about 50%, preferably 90%, and most preferably 95% of the methylethylbenzene to light gas and lighter aromatics. Furthermore, such high ratios are evidence that the reactions do not yield methylethylbenzene.

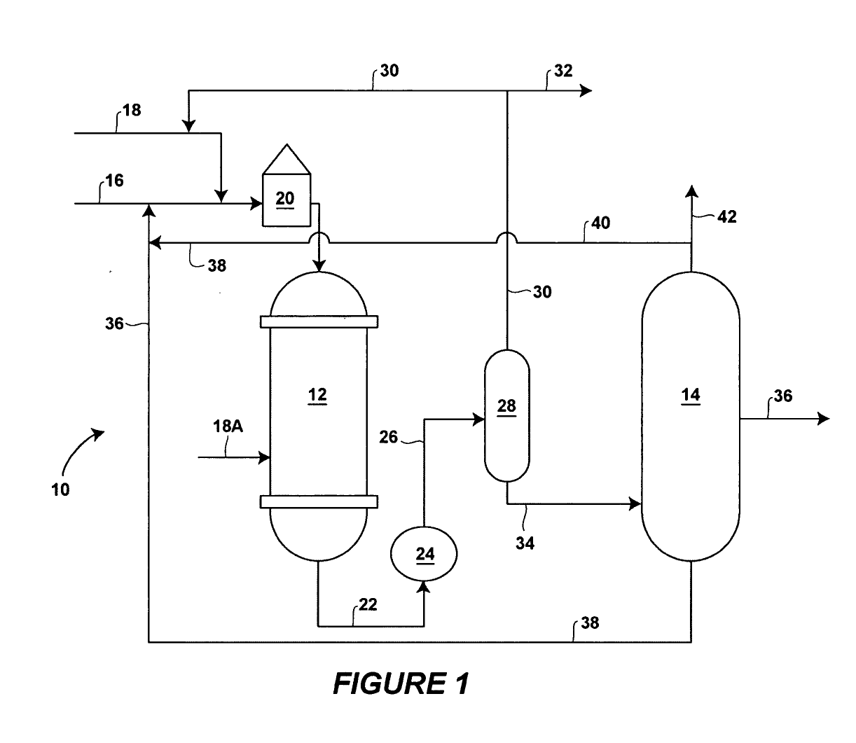

The disclosed process is generally illustrated in FIG. 1, wherein an embodiment, generally designated 10, of the process includes a reactor 12 and a liquid products separator 14. More specifically, a C₉ aromatics-comprising feed in a feed line 16 and a hydrogen-comprising gas in a gas line 18 are combined and heated in a furnace 20. The heated mixture is passed into the reactor 12 where the C₉ aromatics-comprising feed catalytically reacts in the presence of hydrogen to yield an intermediate product. The intermediate product exits the reactor 12 through an intermediate product line 22 and is thereafter cooled in a heat exchanger 24. A cooled, intermediate product exits the heat exchanger 24 via a transport line 26 and passes into a vessel 28 in which gas and liquids are separated from one another. As necessary, fresh hydrogen also can be passed directly into the reactor 12 via a gas line 18A for purposes of cooling the reactor 12. Gases, primarily hydrogen, are withdrawn from the vessel 28, and portions are compressed (compressor not shown), and recycled via a gas line 30 to the hydrogen-comprising gas in line 18, while the remainder may be purged via a purge line 32. The liquids are withdrawn from the vessel 28 via a transport line 34 and passed into the liquids separator 14. Within the separator 14, constituents comprising the intermediate product are separated. A xylene isomers product exits the separator via a conduit 36. One or more recycle streams carry C₉ aromatics (38) and benzene and toluene (40) back to the reactor 12, for example, by combining these streams with fresh feed in the feed line 16. Thus, entering this embodiment 10 of the process are a C₉ aromatics-comprising feed (16) and a hydrogen-comprising gas (18), and exiting the process is a xylene isomers product (36). Because the transalkylation and disproportionation performed in the process require a certain number of methyl groups to be present relative to the number of benzene groups, there may be some bleeding of the formed benzene and toluene (42) out of the overall process, but not to any significant amount. The process also can include the use of recycle streams as described in more detail below.

Subsumed in the disclosed method (and the various embodiments thereof is an understanding by those skilled in the art of suitable processing equipment and controls necessary to carry out the method. Such processing equipment includes, but is not limited to, appropriate piping, pumps, valves, unit operations equipment (e.g., reactor vessels with appropriate inlets and outlets, heat exchangers, separation units, etc.), associated process control equipment, and quality control equipment, if any. Any other processing equipment, especially where particularly preferred, is specified herein.

Generally, the disclosed method is carried out in a reaction vessel containing an active catalyst and, as discussed in more detailed below, such a catalyst comprises a large-pore zeolite impregnated with a Group VIB metal oxide, and a suitable binder. Large pore zeolites suitable for use in accordance with the invention include zeolites having a pore size of at least about 6 angstroms, and include beta (BEA), EMT, FAU (e.g., zeolite X, zeolite Y (USY)), LTL, MAZ, mazzite, mordenite (MOR), omega, SAPO-37, VFI, zeolite L structure type zeolites (IUPAC Commission of Zeolite Nomenclature). Preferably, however, large-pore zeolites for use in the invention include beta (BEA), Y (USY), and mordenite (MOR) zeolites, general descriptions of each of which can be found in Kirk Othmer's “Encyclopedia of Chemical Technology,” 4^{th} Ed., Vol. 16, pp. 888-925 (John Wiley & Sons, New York, 1995) and W. M. Meier et al., “Atlas of Zeolite Structure Types,” 4^{th} Ed. (Elsevier 1996), the disclosures of which are incorporated by reference herein. These types of zeolites can be obtained from commercial sources such as, for example, the PQ Corporation (Valley Forge, Pa.), Tosoh USA, Inc. (Grove City, Ohio), and UOP Inc. (Des Plaines, Ill.). More preferably, the large-pore zeolite for use in the invention is a mordenite zeolite.

Any metal oxide that, when incorporated into a zeolite, is capable of promoting the hydrodealkylation of a C₉₊ aromatic compounds to a C₆ to C₈ aromatic hydrocarbon can be employed in the invention. The metal oxide preferably is selected from the group consisting of molybdenum oxides, chromium oxides, tungsten oxides, and combinations of any two or more thereof wherein the oxidation state of the metal can be any available oxidation state. For example, in the case of a molybdenum oxide, the oxidation state of molybdenum can be 0, 2, 3, 4, 5, 6, or combinations of any two or more thereof.

Examples of suitable metal compounds include, but are not limited to, chromium-, molybdenum-, and/or tungsten-containing compounds. Suitable chromium-containing compounds include, but are not limited to, chromium(II) acetate, chromium(II) chloride, chromium(II) fluoride, chromium(III) 2,4-pentanedionate, chromium(III) acetate, chromium(III) acetylacetonate, chromium(III) chloride, chromium(III) fluoride, chromium hexacarbonyl, chromium(III) nitrate, chromium nitride, chromium(III) perchlorate, and, chromium(III) telluride. Suitable tungsten-containing compounds include, but are not limited to, tungstic acid, tungsten(V) bromide, tungsten(IV) chloride, tungsten(VI) chloride, tungsten hexacarbonyl, and tungsten(VI) oxychloride. Molybdenum-containing compounds are the preferred metal and such compounds include, but are not limited to, ammonium dimolybdate, ammonium heptamolybdate(VI), ammonium molybdate, ammonium phosphomolybdate, ammonium tetrathiomolybdate, ammonium tetrathiomolybdate, bis(acetylacetonate)dioxomolybdenum(VI), molybdenum fluoride, molybdenum hexacarbonyl, molybdenum oxychloride, molybdenum sulfide, molybdenum(I) acetate, molybdenum(I) chloride, molybdenum(II) bromide, molybdenum(III) chloride, molybdenum(IV) chloride, molybdenum(V) chloride, molybdenum(VI) fluoride, molybdenum(VI) oxychloride, molybdenum(VI) tetrachloride oxide, potassium molybdate, sodium molybdate, and molybdenum oxides in which the oxidation state of Mo can be 2, 3, 4, 5, and 6, and combinations of two or more thereof. Preferably, the metal compound is an ammonium molybdate due to its abundance and the relative ease with which molybdenum can be incorporated into the preferred mordenite zeolites.

The amount of metal or metal oxide present in the catalyst composition should be sufficient to be effective with transalkylation and disproportionation processes. Accordingly, the amount of metal or metal oxide preferably is in a range of about 0.1 wt. % to about 40 wt. %, based on the total weight of the catalyst composition, and more preferably about 0.5 wt. % to about 20 wt. %, and even more preferably about 1 wt. % to 10 wt. %. If a combination of metal or metal oxides is used, the molar ratio of the second, third, and fourth metal oxides to the first metal oxide should be in a range of about 0.01:1 to about 100:1.

Molybdenum is the preferred metal and, when present in an amount of about 1 wt. % to about 5 wt. %, results in conversions that are unexpectedly and surprisingly superior to that obtained when the amount falls outside of this range. Such unexpected and surprisingly superior results are shown in the examples, below. In view of these findings, preferably the catalyst is impregregnated with molybdenum or molybdenum oxide, wherein the molybdenum comprises about 0.5 wt. % to about 10 wt. % of the catalyst, based on the total weight of the catalyst. More preferably, the molybdenum comprises about 1 wt. % to about 5 wt. % of the catalyst, and most preferably, the molybdenum comprises about 2 wt. % of the catalyst, based on the total weight of the catalyst.

Suitable binders for use in preparing the catalyst include, but are not limited to, aluminas such as, for example, α-alumina and γ-alumina; silicas; alumina-silica; and combinations thereof. The weight ratio of the zeolite to the binder preferably is about 20:1 to about 0.1:1, and more preferably about 10:1 to about 0.5:1. The binder is typically combined with the zeolite in the presence of a liquid, preferably in an aqueous medium, to form a zeolite-binder mixture.

Any suitable methods for incorporating a metal oxide compound into a zeolite such as, for example, impregnation or adsorption can be used to make a catalyst for use in accordance with the disclosed method. For example, the zeolite and the binder can be well mixed by stirring, blending, kneading, or extrusion, following which the zeolite-binder mixture can be dried in air at a temperature in the range of from about 20° C. to about 200° C., preferably about 25° C. to about 175° C., and more preferably 25° C. to 150° C. for about 0.5 hour to about 50 hours, preferably about one hour to about 30 hours, and more preferably one hour to 20 hours. Preferably, the mixing occurs under atmospheric pressure, but can occur at pressures slightly above and below atmospheric pressure. After the zeolite and binder are sufficiently mixed and dried, the zeolite-binder mixture optionally can be calcined in air at a temperature in a range of about 300° C. to 1000° C., preferably about 350° C. to about 750° C., and more preferably about 450° C. to about 650° C. The calcination can be carried out for about one hour to about 30 hours, and more preferably about two hours to about fifteen hours, to yield a calcined zeolite-binder. If a binder is not desired, a zeolite also can be calcined under similar conditions to remove any contaminants, if present.

The zeolite, with or without a binder, and calcined or not, generally is first mixed, with a metal compound. Where the binder is combined with a metal compound, it can be subsequently converted to a metal oxide by heating at elevated temperature, generally in air. The metal preferably is selected from the Group VIB metals, such as, chromium, molybdenum, tungsten, and combinations thereof as noted above. The metal compound can be dissolved in a solvent before being contacted with the zeolite. Preferably, however, the metal compound is an aqueous solution. The contacting can be carried out at any temperature preferably, however, at a temperature in a range of about 15° C. to about 100° C., more preferably about 20° C. to about 100° C., and even more preferably about 20° C. to 60° C. The contacting generally can be carried out under any pressure, preferably atmospheric pressure, for a length of time sufficient to ensure a mixture of the metal compound and the zeolite. Generally, this length of time is about one minute to about fifteen hours, and preferably about one minute to about five hours.

Depending on the severity of operation and other process parameters, the catalyst will age. As the catalyst ages, its activity for the desired reactions tends to slowly diminish due to the formation of coke deposition or feed poisons on the surfaces of the catalyst. The catalyst may be maintained at or periodically regenerated to its initial level of activity by methods generally known by those of ordinary skill in the art. Alternatively, the aged catalyst may simply be replaced with new catalyst.

To the extent that the aged catalyst is not replaced with new catalyst, the aged catalyst may require regeneration as frequently as once every six months, as often as once every three months, or, on occasion, as often as once or twice every month. As used herein, the term “regeneration” means the recovery of at least a portion of the molecular sieve initial activity by combusting any coke deposits on the catalyst with oxygen or an oxygen-containing gas. The literature is replete with catalyst regeneration methods that can be used in the process of the present invention. Some of these regeneration methods involve chemical methods for increasing the activity of deactivated molecular sieves. Other regeneration methods relate to methods of regenerating coke-deactivated catalysts by the combustion of the coke with an oxygen-containing gas stream such as, for example, a cyclic flow of regeneration gases or the continuous circulation of an inert gas containing a quantity of oxygen in a closed loop arrangement through the catalyst bed.

The catalyst for use in the disclosed method is particularly suited for regeneration by the oxidation or burning of catalyst deactivating carbonaceous deposits (also known as coke) with oxygen or an oxygen-containing gas. Though the methods by which a catalyst may be regenerated by coke combustion can vary, preferably it is performed at conditions of temperature, pressure, and gas space velocity, for example, which are least damaging thermally to the catalyst being regenerated. It is also preferable to perform the regeneration in a timely manner to reduce process down-time in the case of a fixed bed reactor system or equipment size, in the case of a continuous regeneration process.

Though the optimum regeneration conditions and methods are generally known by those having ordinary skill in the art, catalyst regeneration preferably is accomplished at conditions including a temperature range of about 550° F. (about 287° C.) to about 1300° F. (about 705° C.), a pressure range of about zero pounds per square inch gauge (psig) (about zero mega-Pascals (MPa)) to about 300 psig (about two MPa), and a regeneration gas oxygen content of from about 0.1 mole percent to about 25 mole percent. The oxygen content of the regeneration gas typically can be increased during the course of a catalyst regeneration procedure based on catalyst bed outlet temperatures to regenerate the catalyst as quickly as possible while avoiding catalyst-damaging process conditions. The preferred catalyst regeneration conditions include a temperature ranging from about 600° F. (about 315° C.) to about 1150° F. (about 620° C.), a pressure ranging from about zero psig (about zero MPa) to about 150 psig (about one MPa), and a regeneration gas oxygen content of about 0.1 mole percent to about ten mole percent. The oxygen-containing regeneration gas preferably comprises nitrogen and carbon combustion products such as carbon monoxide and carbon dioxide, to which oxygen in the form of air has been added. However, it is possible that the oxygen can be introduced into the regeneration gas as pure oxygen, or as a mixture of oxygen diluted with another gaseous component. Preferably the oxygen-containing gas is air.

As noted above, the disclosed method is carried out in the presence of a hydrogen-containing gas, wherein the gas comprises hydrogen (i.e., molecular hydrogen, H₂). Such hydrogen-containing gas preferably comprises hydrogen in a range of about one volume percent (vol. %) to about 100 vol. %, preferably about 50 vol. % to about 100 vol. %, and more preferably 75 vol. % to 100 vol. %. If the hydrogen content in the gas is less than about 100 vol. %, then the remainder of the gas may be any inert gas such as, for example, nitrogen, helium, neon, argon, and combinations thereof, or any other gas which does not detrimentally affect the disclosed methods and the catalyst used therein. Hydrogen can be supplied from a hydrogen plant, a catalytic reforming facility, or other hydrogen-producing or hydrogen-recovery processes.

Hydrogen preferably is present during the catalytic reaction in a hydrogen-to-hydrocarbon molar ratio of about 0.01 to about five, more preferably about 0.1 to about two, and more preferably about 0.1 to about 0.5. Hydrogen circulation rates below these ranges can result in higher catalyst deactivation rates resulting in increased and more frequent energy intensive regeneration cycles. Excessively high reaction pressures increase energy and equipment costs and provide diminishing marginal benefits. Excessively high hydrogen circulation rates also can influence reaction equilibrium and drive the reaction undesirably towards reduced C₉ aromatics conversion and lower xylene isomers yield, for example. The presence of inert gases can beneficially serve to reduce the partial pressure of the hydrocarbon resulting in higher conversions of the feedstock to xylene isomers.

The contacting of a fluid feed stream containing a hydrocarbon with a hydrogen-containing fluid (gas or liquid) in the presence of the catalyst composition can be carried out in any technically suitable manner, in a batch or semi-continuous or continuous process, under a condition effective to convert a hydrocarbon to a C₆ to C₈ aromatic hydrocarbon. Generally, a fluid stream as disclosed above, preferably being in the vaporized state, is introduced with the feed into a suitable hydroprocessing reactor having a fixed catalyst bed, or a moving catalyst bed, or a fluidized catalyst bed, or combinations of any two or more thereof by any means known to one skilled in the art such as, for example, pressure, meter pump, and other similar means. Because a hydroprocessing reactor and process therewith are well known to one skilled in the art, its description is omitted herein in the interest of brevity.

Conditions suitable for carrying out the process of the invention can include a weight hourly space velocity (WHSV) of the fluid feed stream in the range of about 0.1 to about 20, preferably about 0.5 to about 10, and most preferably about 1 to about 5 unit mass of feed per unit mass of catalyst per hour. The hydrogen-containing fluid (gas) hourly space velocity generally is in the range of about 1 to about 10,000, preferably about 5 to about 7,000, and most preferably about 10 to about 10,000 ft³ H₂/ft³ catalyst/hour.

Generally, the pressure can be in a range of about 0.5 MPa (about 73 psig) to about 5 MPa (about 725 psig), preferably about 1 MPa (about 145 psig) to about 3 MPa (about 435 psig), and more preferably about 1.25 MPa (about 181 psig) to about 2 MPa (about 190 psig). The temperature suitable for carrying out the process of the invention is in a range of about 200° C. (about 392° F.) to about 1000° C. (about 1830° F.), more preferably about 300° C. (about 572° F.) to about 800° C. (about 1472° F.), and even more preferably about 350° C. (about 662° F.) to about 600° C. (about 1112° F.).

### EXAMPLES

The following examples are provided to illustrate the invention, but are not intended to limit the scope thereof. Example 1 is directed to the preparation of catalysts which were then used in the processes described in Examples 2 through 4. Example 3-A is based on process modeling using the feed described in Example 3 and catalyst “A”, whereas Example 3-B is based on similar process modeling using the feed described in Example 3 and catalyst “B.” Example 5 illustrates the performance capabilities of large-pore, molybdenum-impregnated zeolite catalysts.

### Example 1

This example describes the preparation of two catalysts (Catalysts “A” and “B”), which were subsequently used in the processes described in Examples 2 through 4. A first catalyst, Catalyst “A,” is a mordenite zeolite, whereas the second catalyst, Catalyst “B” comprises a molybdenum-impregnated, mordenite zeolite. This example also describes the preparation of two other catalysts (Catalysts “C” and “D”), which were subsequently used in the process described in Example 5. Catalyst “C” comprised a molybdenum-impregnated, beta zeolite, while Catalyst “D” comprised a molybdenum-impregnated, USY zeolite

More specifically, catalyst “A” was a mordenite zeolite that was prepared by mixing 80 grams of H-mordenite zeolite (commercially-available from Union Carbide Corporation (Houston, Tex.) under the tradename “LZM-8”) with 100 grams of distilled water and 215 grams of Al₂O₃ sol (9.3% solid in water) (commercially available as Alumina sol from Criterion). The mixture was then dried at 329° F. (165° C.) for about three hours and thereafter calcined at 950° F. (510° C.) for about four hours to obtain a mordenite catalyst (80% sieve/20% Al₂O₃). After calcination, the catalyst was granulated and passed through 14/40 sieves.

Catalyst “B” was a molybdenum-impregnated mordenite (MOR) catalyst (i.e., 2% Mo/MOR catalyst). Specifically, 1.32 grams of ammonium heptamolybdate ((NH₄)₆Mo₇O₂₄.4H₂O) was dissolved into 32 grams of distilled water to achieve a clear solution. The clear solution was then added to and mixed with 36 grams of the catalyst “A” (prepared as described above), dried at 329° F. (165° C.) for about three hours, and thereafter calcined at 950° F. (510° C.) for about four to obtain the impregnated catalyst (i.e., Catalyst “B”).

Catalyst “C” was a molybdenum-impregnated beta (BEA) zeolite (i.e., 2% Mo/BEA catalyst). The beta catalyst (80% sieve/20% Al₂O₃) was prepared by mixing 64 grams of H-β Zeolite (commercially-available from PQ Corporation (Valley Forge, Pa.)) with 22 grams of distilled water and 172 grams of Al₂O₃ sol (9.3% solid in water) (commercially available as Alumina sol from Criterion). The mixture was then dried at 329° F. (165° C.) for about three hours, and thereafter calcined at 950° F. (510° C.) for about four hours. After calcination, the catalyst was granulated and passed through 14/40 sieves. An aqueous solution of ammonium heptamolybdate containing 0.784 grams was mixed with 21.3 grams of the prepared beta catalyst, dried at 329° F. (165° C.) for about three hours, and thereafter calcined at 950° F. (510° C.) for about four hours to obtain the impregnated catalyst (i.e., Catalyst “C”).

Catalyst “D” was a molybdenum-impregnated USY zeolite (i.e., 5% Mo/USY catalyst). The USY catalyst (80% sieve/20% Al₂O₃) was prepared by mixing 80 grams of H-USY zeolite (commercially available from UOP, Inc. (Des Plaines, Ill.), under the tradename “LZY-84”) with 215 grams of Al₂O₃ sol (9.3% solid in water) (commercially available as Alumina sol from Criterion). The mixture was then dried at 329° F. (165° C.) for about three hours, and thereafter calcined at 950° F. (510° C.) for about four hours. After calcination, the catalyst was granulated and passed through 14/40 sieves. An aqueous solution of ammonium heptamolybdate containing 2.35 grams was mixed with 25 grams of the prepared USY catalyst, dried at 329° F. (165° C.) for about three hours, and thereafter calcined at 950° F. (510° C.) for about four to obtain the impregnated catalyst (i.e., Catalyst “D”).

### Example 2

This example illustrates the performance capabilities of a mordenite catalyst (Catalyst “A” of Example 1) and an identical catalyst impregnated with molybdenum (Catalyst “B” of Example 1) to convert nitration-grade toluene to benzene and xylenes. In each run, the ground catalyst was packed into a ¾-inch tubular, stainless steel, plug-flow reactor and treated with flowing hydrogen for two hours at 400° C. (752° F.) and 200 pounds per square inch gauge (psig) (about 1.4 megapascals (MPa)) prior to the introduction of the liquid feed. The feed stream was a mixture of hydrogen and toluene (4:1 hydrogen:toluene molar ratio), and the reaction conditions were 400° C. (752° F.) and 200 psig (about 1.4 MPa), and at a WHSV of 1.0 and 2.0 for catalyst “A”, and 1.0, 2.0 and 5.0 for catalyst “B”. Analyses of the liquid feeds (Feed Wt. %) and products (Pdt. Wt. %) obtained in each run are shown in Table 1.

TABLE 1

| | Feed | Catalyst “A” | | Catalyst “B” | | |
|---|---|---|---|---|---|---|
| | Wt % | Pdt. Wt % | | Pdt. Wt. % | | |
| WHSV | | 1.0 | 2.0 | 1.0 | 2.0 | 5.0 |
| Light Gas | 0.01 | 0.54 | 0.38 | 3.48 | 2.90 | 0.87 |
| Benzene | 0.00 | 17.51 | 13.59 | 23.29 | 23.35 | 13.48 |
| Toluene | 99.76 | 58.49 | 65.06 | 43.30 | 44.17 | 66.93 |
| Ethylbenzene | 0.05 | 0.36 | 0.38 | 0.33 | 0.32 | 0.21 |
| p-Xylene | 0.04 | 4.86 | 4.20 | 5.78 | 5.72 | 4.01 |
| m-Xylene | 0.06 | 10.58 | 9.12 | 12.67 | 12.51 | 8.70 |
| o-Xylene | 0.00 | 4.65 | 3.97 | 5.55 | 5.49 | 3.80 |
| Propylbenzene | 0.00 | 0.01 | 0.01 | 0.01 | 0.01 | 0.00 |
| Methylethylbenzene | 0.00 | 0.65 | 1.10 | 0.34 | 0.40 | 0.55 |
| Trimethylbenzene | 0.00 | 2.18 | 1.91 | 4.76 | 4.70 | 1.32 |
| A₁₀₊ | 0.09 | 0.18 | 0.17 | 0.46 | 0.44 | 0.13 |

The conversion of toluene is determined by dividing the difference in the amount of toluene in the feed and product by the toluene present in the feed. For example, using the data obtained from the run with catalyst “A” and a WHSV of 2.0, the toluene conversion was about 34.8 (i.e., 34.8=100×(99.76−65.06)+99.76). The selectivity of any particular constituent in the product is determined by dividing the yield of the constitutent by the conversion of toluene. Thus, for example, using the data obtained from the run with catalyst “A” and a WHSV of 2.0, the benzene selectivity was about 39% (i.e., 39=100×(13.59−34.8)), and the xylene isomers selectivity was about 49.7% (i.e., 49.7=100×17.29+34.8)).

For catalyst “B”, the conversion is nearly identical at WHSV 1 and 2, indicating that the catalyst is near equilibrium conversion. The data show that an increase in the WHSV results in lower conversion of toluene (from 57% to 56% to 33% for WHSVs of 1, 2, and 5, respectively) when using catalyst “B”. This trend is also shown by the data when using catalyst “A” (from 41% to 35% for WSHVs of 1 and 2, respectively). Based on the profiles of the products produced using each catalyst, it is readily seen that the addition of 2 wt % molybdenum oxide generally did not significantly affect the production of one particular constituent (selectivity) over another. At WHSV of 5, the benzene and xylene selectivity obtained using catalyst “B” are 40.8 and 49.8 respectively, and very similar to that obtained when using catalyst “A.” The addition of 2% molybdenum oxide resulted in an increased catalyst activity by about 2.5 times compared to catalyst “A.” The yield of by-product light gas is higher and heavy aromatics are lower giving a slightly higher yield of less desirable products.

### Example 3

This example illustrates the performance capabilities of a mordenite catalyst (Catalyst “A” of Example 1) and an identical catalyst impregnated with molybdenum (Catalyst “B” of Example 1) to convert a near 100% C₉ aromatics-comprising feed to xylene isomers. The composition of the feed is provided in Table 2, below, and was identical in each of the five runs. In each run, the catalyst was packed into a %-inch tubular, stainless steel, plug-flow reactor and treated with flowing hydrogen for two hours at 400° C. (752° F.) and 200 psig (about 1.4 MPa) prior to the introduction of the liquid feed. The feed stream was a mixture of hydrogen and hydrocarbon in a 4:1 molar ratio, and the reaction conditions were 400° C. (752° F.), 200 psig (about 1.4 MPa). The WHSV for the two runs using catalyst A were 1.0 and 1.5, while the WHSV for the three runs using catalyst “B” was 1.0, 1.5, and 2.0. Analyses of the liquid feeds and products obtained in each run are shown in Table 2, below.

TABLE 2

| | Feed | Catalyst “A” | | Catalyst “B” | | |
|---|---|---|---|---|---|---|
| | Wt % | Pdt. Wt % | | Pdt. Wt. % | | |
| WHSV | | 1.0 | 1.5 | 1.0 | 1.5 | 2.0 |
| Light Gas | 0.20 | 2.86 | 2.04 | 12.48 | 9.09 | 10.61 |
| Benzene | 0.12 | 2.09 | 1.98 | 5.15 | 4.90 | 4.47 |
| Toluene | 0.01 | 9.81 | 7.84 | 23.44 | 23.50 | 21.51 |
| Ethylbenzene | 0.05 | 3.05 | 2.55 | 0.52 | 0.89 | 1.69 |
| p-Xylene | 0.19 | 1.91 | 1.31 | 8.38 | 8.53 | 7.96 |
| m-Xylene | 0.47 | 4.05 | 2.76 | 18.28 | 18.72 | 17.34 |
| o-Xylene | 0.32 | 1.90 | 1.38 | 8.01 | 8.18 | 7.55 |
| Propylbenzene | 6.62 | 0.69 | 1.26 | 0.00 | 0.00 | 0.00 |
| Methylethylbenzene | 49.32 | 30.67 | 35.00 | 1.31 | 2.19 | 4.07 |
| Trimethylbenzene | 41.77 | 33.40 | 35.80 | 18.67 | 19.50 | 19.06 |
| A₁₀₊ | 0.94 | 9.59 | 8.08 | 3.76 | 4.55 | 5.60 |

Based on the data shown in Table 2, above, there are unexpected and surprising results obtained when using catalyst “B.” For example, surprisingly and unexpectedly high conversion of the feed is obtainable with catalyst “B” when compared to catalyst “A.” Specifically, the liquid product obtained when using catalyst “A” has a weight ratio of C₉ aromatics present in the feed to that present in the product of about 1.51 (i.e., 97.71/64.76) at WHSV of 1.0, and 1.35 (i.e., 97.71/72.06) at WHSV of 1.5. In contrast, the liquid product obtained when passing an identical feed under identical reaction conditions, but using catalyst “B,” has a weight ratio of C₉ aromatics present in the feed to that present in the product of about 4.89 (i.e., 97.71/19.98) at WHSV of 1.0, and 4.5 (i.e., 97.71/21.69) at WHSV of 1.5. This unexpected and surprisingly high conversion is beneficial in that there are lower amounts of unreacted C₉ aromatics that need to be recycled back to the reactor for conversion. Though the addition of molybdenum is expected to increase the longevity (activity) of the catalyst, it is unexpected and surprising that the addition of the molybdenum results in such a high conversion of the C₉ aromatics to xylene isomers.

Furthermore, surprisingly and unexpectedly high conversion of the C₉ aromatics to xylene isomers is obtainable with catalyst “B” when compared to catalyst “A.” Specifically, the liquid product obtained when using catalyst “A” has a weight ratio of xylene isomers to C₉ aromatics of about 0.12 (i.e., 7.86/64.76) at WHSV of 1.0, and 0.08 (5.45/72.06) at WSHV of 1.5. In contrast, the liquid product obtained when passing the identical feed under identical reaction conditions, but using catalyst “B,” has a weight ratio of xylene isomers to C₉ aromatics of about 1.74 (i.e., 34.67/19.98) at WHSV 1.0, and 1.63 (35.43/21.69) at WHSV of 1.5.

Similarly, the data in Table 2 show surprisingly and unexpectedly high conversion of the methylethylbenzene with catalyst “B” when compared to catalyst “A.” Specifically, the liquid product obtained when using catalyst “A” has a weight ratio of methylethylbenzene present in the feed to that present in the product of about 1.61 (i.e., 49.32/30.67) at WHSV of 1.0, and 1.41 (i.e., 49.32/35) at WHSV of 1.5. In contrast, the liquid product obtained when passing an identical feed under identical reaction conditions, but using catalyst “B,” has a weight ratio of methylethylbenzene present in the feed to that present in the product of about 37.65 (i.e., 49.32/1.31) at WHSV of 1.0, and 22.58 (i.e., 49.32/2.19) at WHSV of 1.5. This unexpected and surprisingly high conversion is beneficial in that there are lower amounts of unreacted methylethylbenzene that need to be recycled back to the reactor for conversion.

Still further, the liquid product obtained when using catalyst “A” has a weight ratio of xylene isomers to ethylbenzene of about 2.58 (i.e., 7.86/3.05) at WHSV of 1.0, and 2.14 (i.e., 5.45/2.55) at WHSV of 1.5. In contrast, the liquid product obtained when passing a substantially identical feed under identical reaction conditions, but using catalyst “B,” has a weight ratio of xylene isomers to ethylbenzene is about 66.67 (i.e., 34.67/0.52) at WHSV of 1.0, and 39.81 (i.e., 35.43/0.89) at WHSV of 1.5. This unexpected and surprisingly high weight ratio is beneficial in downstream processing where, as described above, the product stream is to be fractionated into its major constituents, i.e., into aromatics containing 6, 7, 8, and 9 carbons. Typically, further processing of a C₈ aromatics fraction would necessarily involve energy-consuming processing of the ethylbenzene. However, given the substantial absence of ethylbenzene in the liquid reaction product obtained when using catalyst “B,” and the accordingly substantial absence of ethylbenzene in the C₈ aromatics fraction, no such energy-consuming processing is required to rid the fraction of ethylbenzene. This is but one of the benefits realized by the use of catalyst “B” versus catalyst “A” under the given reaction conditions and given feed.

Additionally, the product obtained with catalyst “B” has a surprisingly and unexpectedly high weight ratio of xylene isomers to C₁₀ aromatics in comparison to the product obtained using catalyst “A.” Specifically, the liquid product obtained when using catalyst “A” has a weight ratio of xylene isomers to C₁₀ aromatics of about 0.82 (i.e., 7.86/9.59) at WHSV of 1.0, and 0.67 (i.e., 5.45/8.08) at WHSV of 1.5. In contrast, the liquid product obtained when passing an identical feed under identical reaction conditions, but using catalyst “B,” has a weight ratio of xylene isomers to C₁₀ aromatics of about 9.22 (i.e., 34.67/3.76) at WHSV of 1.0, and 7.79 (i.e., 35.43/4.55) at WHSV of 1.5. Such high ratios are evidence that the dominant reaction involving theC₉ aromatics is a disproportionation reaction resulting in xylene isomers and not a reaction yielding C₁₀ aromatics and benzene. Again, the lack of or low amounts of C₁₀ aromatics in the product and/or intermediate product stream is advantageous in that the there are lower amounts of such unreacted or produced C₁₀ aromatics that need to be recycled back to the feed for conversion, thus, conserving energy and reducing capital costs. To the extent that C₁₀ aromatics are present in the intermediate or product stream, such C₁₀ aromatics are predominantly tetramethylbenzene, which can be recycled and are more amenable to conversion to xylene isomers. Advantageously, and in contrast to the product obtained with catalyst “A,” the C₁₀ aromatics present in the product obtained from catalyst “B” do not include ethyldimethylbenzene and/or diethylbenzene, both of which are more difficult to convert to xylene isomers and, therefore, less amenable to recycle.

The product obtained with catalyst “B” also has a surprisingly and unexpectedly high weight ratio of trimethylbenzene to methylethylbenzene in comparison to the product obtained using catalyst “A.” Specifically, the liquid product obtained when using catalyst “A” has a weight ratio of trimethylbenzene to methylethylbenzene of about 1.1 (i.e., 33.4/30.67) at WHSV of 1.0, and 1.0 (i.e., 35.8/35.0) at WHSV of 1.5. In contrast, the liquid product obtained when passing an identical feed under identical reaction conditions, but using catalyst “B,” has a weight ratio of trimethylbenzene to methylethylbenzene of about 14.25 (i.e., 18.67/1.31) at WHSV of 1.0, and 8.9 (i.e., 19.5/2.19) at WHSV of 1.5. This unexpected and surprisingly high ratio is beneficial because trimethylbenzene is more easily convertible to xylene isomers than is methylethylbenzene and, consequently, is more amenable to recycle.

Still further, the product obtained with catalyst “B” has a surprisingly and unexpectedly high weight ratio of benzene to ethylbenzene in comparison to the product obtained using catalyst “A.” Specifically, the liquid product obtained when using catalyst “A” has a weight ratio of benzene to ethylbenzene of about 0.69 (i.e., 2.09/3.05) at WHSV of 1.0, and 0.78 (i.e., 1.98/2.55) at WHSV of 1.5. In contrast, the liquid product obtained when passing an identical feed under identical reaction conditions, but using catalyst “B,” has a weight ratio of benzene to ethylbenzene of about 9.9 (i.e., 5.15/0.52) at WHSV of 1.0, and 5.51 (i.e., 4.9/0.89) at WHSV of 1.5.

The results shown in Table 2, above, with respect to toluene disproportionation, illustrate that addition of 2% molybdenum oxide increases the activity of the catalyst, as evidenced by the higher methyethylbenzene and trimethylbenzene conversions under identical conditions. Referring back to the results obtained in Example 2, above, the selectivities obtained using catalyst “A” and “B” for toluene disproportionation were nearly identical or slightly worse for catalyst “B.” The data reported in Table 3, below, show that for conversion of C₉ aromatics, the selectivity to xylenes is significantly higher, the selectivity to benzene is slightly lower, and the selectivity to heavy C₁₀₊ aromatics is significantly lower.

TABLE 3

| | Catalyst “A” | | Catalyst “B” | | |
|---|---|---|---|---|---|
| WHSV | 1.0 | 1.5 | 1.0 | 1.5 | 2.0 |
| A₉ Conversion | 32.9 | 21.2 | 79.6 | 77.8 | 76.3 |
| Selectivity for Xylenes | 24.0 | 21.2 | 43.6 | 45.5 | 42.7 |
| Selectivity for Benzene | 6.4 | 7.8 | 6.5 | 6.2 | 5.9 |
| Selectivity for A₁₀⁺ | 29.2 | 31.6 | 4.7 | 5.6 | 7.3 |
| Selectivity for Ethylbenzene in A₈ | 28.0 | 23.8 | 1.5 | 2.5 | 4.9 |

With catalyst “B” the amount of ethylbenzene present in the C₈ aromatics fraction is significantly lower than the amount that is present in the same fraction obtained with catalyst “A.” Thus, the C₈ aromatics fraction obtained using catalyst “B” is much better suited as a chemical feedstock for the production of para-xylene. It was found that the heavy C₁₀₊ aromatics present in the product stream obtained using catalyst “B” can be recycled to the process to produce additional xylenes. In contrast, the heavy C₁₀₊ aromatics present in the product stream obtained using catalyst “A” could not be so recycled because this fraction contained particular C₁₀₊ aromatics (e.g., ethyldimethylbenzene and diethylbenene) that are not easily converted to xylene isomers and would rapidly deactivate the catalyst. When using catalyst “A,” much of the methylethylbenzene reacts to form diethyl-C₁₀₊ aromatics and toluene or ethyldimethylbenzene and ethylbenzene. When using catalyst “B,” however, methylethylbenzene dealkylates the ethyl groups and saturates the groups to yield ethane with production of toluene. Little ethylbenzene is formed and the toluene reacts with trimethylbenzene also present in the feed to produce two xylene molecules. The heavy aromatics are an equilibrium distribution of tetramethylbenzenes, which cleanly react with toluene to give additional xylene isomers.

### Example 3-A (Steady-State Operation with Catalyst “A”)

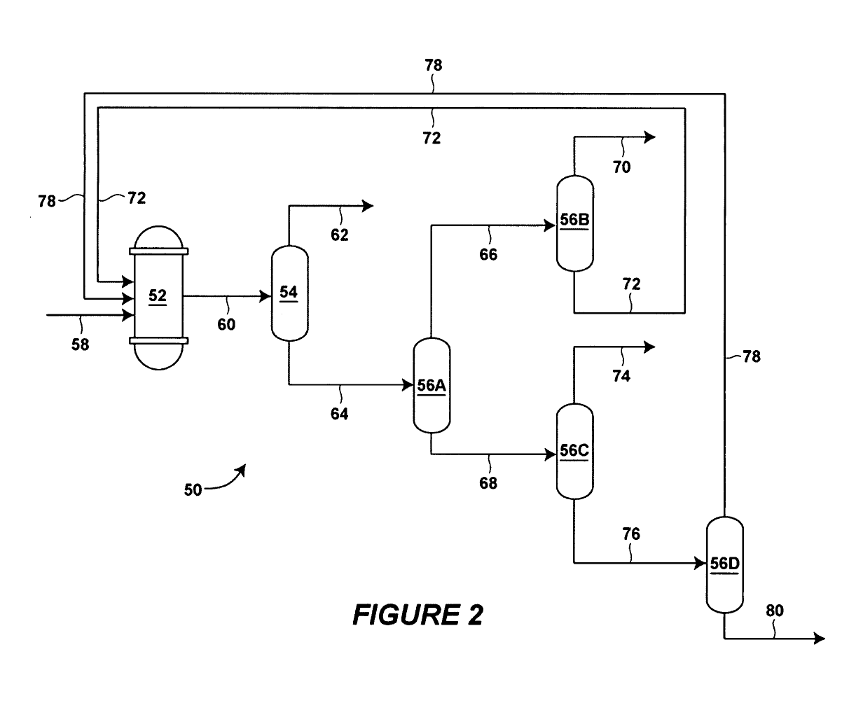

The foregoing example shows the conversion obtainable in a single pass. It is also possible to determine or estimate the conversion obtainable in a steady state process using recycle. The recycle yield in the process using catalyst “A” was determined by process modeling based on the results set forth in Table 2, above. The process flow diagram based on this modeling is shown in FIG. 2.

With reference to FIG. 2, the process flow, generally designated 50, includes a reactor 52 and a distillation train defined by a liquids product separator 54 and multiple distillation columns 56A, 56B, 56C, and 56D. Generally, a C₉ aromatics-comprising feed and hydrogen gas are passed through a line 58 and into the reactor 52 where the feed catalytically reacts (catalyst “A”) in the presence of the hydrogen gas to yield an intermediate product, which exits the reactor 52 through an intermediate product line 60 and subsequently enters the liquid products separator 54. The separator 54, in turn, separates the light hydrocarbons (typically gas) from the aromatics (typically liquid), with the light hydrocarbons exiting the process flow via a line 62 and the aromatics exiting the separator 54 via a line 64 and into the first distillation column 56A wherein the aromatics are separated into two fractions, one of which contains predominantly benzene and toluene and the other of which contains higher aromatics (including xylenes). The fraction containing benzene and toluene exits the distillation column 56A via a line 66 and is passed into the second distillation column 56B, while the higher aromatics fraction exits the distillation column 56A via a line 68 and is passed into a third distillation column 56C. The second distillation column 56B separates the incoming feed into fractions containing predominantly benzene 70 and toluene 72. While both fractions may ultimately be recycled, thereby obviating the second distillation column altogether, as shown, only the toluene fraction 72 (which may contain some benzene) is recycled. The third distillation column 56C separates its incoming feed into fractions containing predominantly the desired xylene isomers product 74 and C₉₊ aromatics 76. In turn, the C₉₊ aromatics fraction 76 is fed to the fourth distillation column 56D wherein its feed is separated into a recyclable fraction 78 of unreacted C₉ aromatics, and a heavy C₁₀₊ aromatics by-product fraction 80 (typically containing a mixture of multiply substituted methyl and ethyl aromatics).

Referring back to Table 2, above, for catalyst “A” at 1.0 WHSV, the selectivity of methyl groups in the C₉ feed to non-C₉ product is as follows: 6% to light non-aromatics; 26% to toluene; 36% to xylene; and, 32% to C₁₀₊ heavy aromatics. As both light non-aromatics and C₁₀₊ heavy aromatics are not recycled in the process flow 50 shown in FIG. 2, these fractions are unavailable for eventually being converted into mixed xylenes. The selectivity of aromatic rings in the C₉ feed to non-C₉ product is as follows: 69% to BTX; 10% to ethylbenzene; and, 21% to C₁₀₊ heavy aromatics. Assuming 100 pounds (lbs.) of C₉ feed, then there would be 1.49 pound-moles (lbmoles) of methyl groups in the feed and 0.822 lbmoles of aromatic rings in the feed. The next step is to calculate whether the availability of methyl or benzyl groups limits the production of xylene isomers. The xylene potential of the methyl groups is determined by multiplying the molar amount of methyl groups available in the feed with the average sum total of selectivity of those methyl groups relative to the toluene and xylenes produced:
1.49 lbmoles×(0.26+0.36)+2=0.462 lbmoles.

Similarly, the xylene potential of the benzyl groups is determined by multiplying the molar amount of benzyl groups by the selectivity of aromatic rings in the feed to BTX in the product:
0.822 lbmoles×0.69=0.567 lbmoles.

Based on the foregoing, it the availability of methyl groups limits the production of xylenes. On this basis, the recycle yield on a molar basis is calculated to be: 0.462 lbmoles xylenes; 0.105 lbmoles benzene (the difference between 0.567 and 0.462); 0.082 lbmoles ethylbenzene; and 0.173 bmoles C₁₀₊ heavies. On a relative weight basis, including light non-aromatics, this becomes: 9% light non-aromatics; 8% benzene; 49% xylenes; 9% ethylbenzene; and, 25% C_{10 +} heavy aromatics.

### Example 3-B (Steady State Operation with Catalyst “B”)

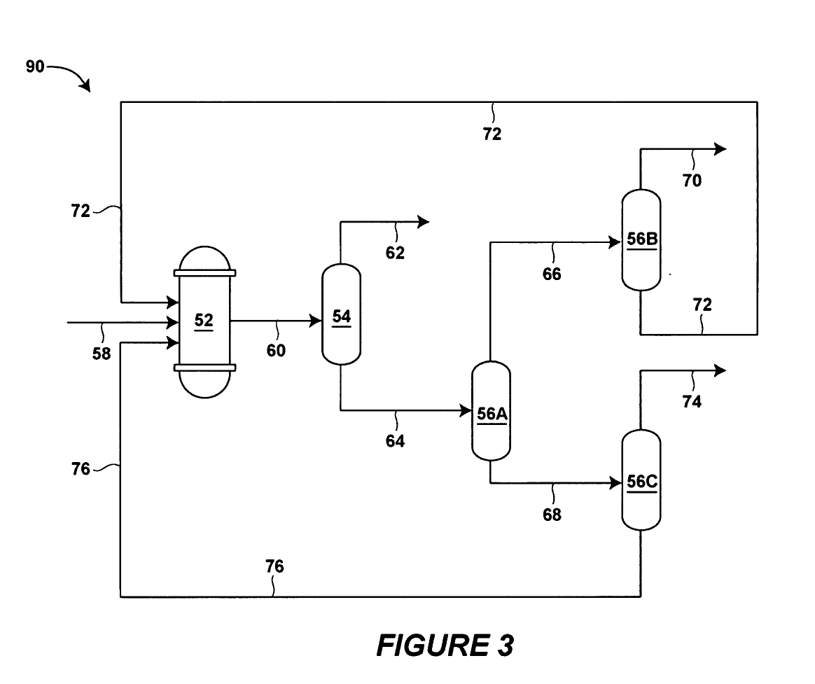

The recycle yield in a steady state process using catalyst “B” was similarly determined by process modeling based on the results set forth in Table 2, above. The process flow diagram based on this modeling is shown in FIG. 3, which bears many resemblances to the modeling shown in FIG. 2, with the exception that different conversions are obtainable.

With reference to FIG. 3, the process flow, generally designated 90, includes the reactor 52 and a distillation train defined by the liquids product separator 54 and multiple distillation columns 56A, 56B, and 56C. Generally, a C₉ aromatics-comprising feed and hydrogen gas are passed through a line 58 and into the reactor 52 where the feed catalytically reacts (catalyst “B”) in the presence of the hydrogen gas to yield an intermediate product—an intermediate product different from that obtained using catalyst “A.” This intermediate product exits the reactor 52 through an intermediate product line 60 and subsequently enters the liquid products separator 54. The separator 54, in turn, separates the light hydrocarbons (typically gas) from the aromatics (typically liquid), with the light hydrocarbons exiting the process flow via a line 62 and the aromatics exiting the separator 54 via a line 64 and into the first distillation column 56A. Therein, the aromatics are separated into two fractions, one of which contains predominantly benzene and toluene and the other of which contains higher aromatics (including xylenes). The fraction containing benzene and toluene exits the distillation column 56A via a line 66 and is passed into the second distillation column 56B, while the higher aromatics fraction exits the distillation column 56A via a line 68 and is passed into a third distillation column 56C. The second distillation column 56B separates its incoming feed into fractions containing predominantly benzene 70 and toluene 72. While both fractions may ultimately be recycled, thereby obviating the second distillation column altogether, as shown, only the toluene fraction 72 (which may contain some benzene) is recycled. The third distillation column 56C separates its incoming feed into a fraction 74 containing the desired xylene isomers product and a fraction 76 containing C₉₊ aromatics, which is recycled to the reactor 52.

Referring back to Table 2, above, for catalyst “B” at 1.0 WHSV, the selectivity of methyl groups in the C₉ feed to non-C₉ product is as follows: 0% to light non-aromatics; 25% to toluene; 65% to xylene; and, 11% to C₁₀₊ heavy aromatics. As C₁₀₊ heavy aromatics are all methyl substituted, they will continue to react with benzene and toluene to produce xylenes. There are no methyl groups lost as by-product in this process flow 90. The selectivity of benzyl groups in the C₉ feed to non-C₉ product is as follows: 96% to BTX; 1% to ethylbenzene; and, 3% to C₁₀₊ heavy aromatics. Assuming, again, 100 lbs of C₉ feed, then there would be 1.49 lbmoles of methyl groups in the feed and 0.822 lbmoles of benzyl groups in the feed. The next step is to calculate whether the availability of methyl or benzyl groups limits the production of xylene isomers. Such calculations are carried out in the manner described above in Example 3-A. The xylene potential of the methyl groups is 0.745 lbmoles, whereas the xylene potential of the benzyl groups is 0.814 lbmoles. Based on the foregoing, it the availability of methyl groups limits the production of xylenes. On this basis, the recycle yield on a molar basis is calculated to be: 0.745 lbmoles xylenes; 0.069 lbmoles benzene (the difference between 0.814 and 0.745); and, 0.008 lbmoles ethylbenzene. On a relative weight basis, including light non-aromatics, this becomes: 15% light non-aromatics; 5% benzene; 79% xylenes; 1% ethylbenzene; and, 0% C₁₀₊ heavy aromatics.

A comparison of the recycle yields obtained in Examples 3-A and 3-B is summarized in Table 4, below.

TABLE 4

| Recycle Yields (%) | Catalyst “A” | Catalyst “B” |
|---|---|---|
| Light Gas | 9 | 15 |
| Benzene | 8 | 5 |
| Xylenes | 49 | 79 |
| Ethylbenzene | 9 | 1 |
| C₁₀₊ Heavies | 25 | 0 |
| % EB in C₈ Aromatics | 15.5 | 1.3 |

### Example 4

This example illustrates the performance capabilities of a mordenite catalyst (Catalyst “A” of Example 1) and an identical catalyst impregnated with molybdenum (Catalyst “B” of Example 1) to convert a feed comprising about 61 wt % C₉ aromatic (A₉) hydrocarbons and about 38 wt % toluene to xylene isomers. Two separate runs were performed with identical feeds. In each run, the catalyst was packed into a ¾-inch tubular, stainless steel, plug-flow reactor and treated with flowing hydrogen for two hours at 400° C. (752° F.) and 200 psig (about 1.4 MPa) prior to the introduction of the liquid feed. The feed stream was a mixture of hydrogen and hydrocarbon in a 4:1 molar ratio, and the reaction conditions were set at 400° C. (752° F.), 200 psig (about 1.4 MPa), and a WHSV of 1.0. Analyses of the liquid feed and product are shown in Table 5, below.

TABLE 5

| | Feed | Catalyst “A” | Catalyst “B” |
|---|---|---|---|
| | Wt % | Pdt. Wt % | Pdt. Wt. % |
| Light Gas | 0.19 | 2.99 | 10.30 |
| Benzene | 0.18 | 3.43 | 11.33 |
| Toluene | 37.51 | 34.64 | 32.12 |
| Ethylbenzene | 0.04 | 3.00 | 0.55 |
| p-Xylene | 0.11 | 3.45 | 7.70 |
| m-Xylene | 0.28 | 7.25 | 16.87 |
| o-Xylene | 0.19 | 3.23 | 7.33 |
| Propylbenzene | 3.99 | 0.26 | 0.00 |
| Methylethylbenzene | 30.75 | 18.02 | 0.93 |
| Trimethylbenzene | 26.08 | 18.89 | 11.29 |
| A₁₀₊ | 0.54 | 4.83 | 1.58 |

The reaction conditions for this example are identical to the conditions used in Example 3. Thus, it is readily apparent that a mixed toluene/C9 aromatics feed reacts under the same processing conditions and, therefore, to the extent one were to start with a pure C9 aromatics feed and produced toluene, such toluene can be recycled to the process for additional production of xylene. Upon operation of recycle, the only products are light gas, benzene and xylene. While both catalysts can convert toluene and C9 aromatics simultaneously, for catalyst “A,” the reaction of toluene and C9 aromatics yields a C8 aromatic product disadvantageously high in ethylbenzene—about 17.8% (i.e., 17.8=100×(3.00/(3.00+3.45+7.25+3.23)). Thus, while processing C9 aromatics together with toluene can produce additional xylenes, the quality of the xylenes for use as a chemical feedstock in the production of para-xylene is poor, i.e., the xylenes produced from the toluene are of a much lower quality compared to the xylenes produced by toluene disproportionation. However, the C8 aromatics produced from an identical feed using catalyst “B” are advantageously, unexpectedly, and surprisingly low in ethylbenzene—about 1.7% (i.e., 1.7%=100×(0.55/(0.55+7.70+16.87+7.33))—thus, resulting in a more high quality xylene product better suited as a chemical feedstock in the production of para-xylene.

Furthermore, there are many other unexpected and surprising results obtained when using catalyst “B.” For example, surprisingly and unexpectedly high conversion of the C₉ aromatics to xylene isomers is obtainable with catalyst “B” when compared to catalyst “A.” Specifically, the liquid product obtained when using catalyst “A” has a weight ratio of C₉ aromatics present in the feed to that present in the product of about 1.64 (i.e., 60.82/37.17). In contrast, the liquid product obtained when passing an identical feed under identical reaction conditions, but using catalyst “B,” has a weight ratio of C₉ aromatics present in the feed to that present in the product of about 4.98 (i.e., 60.82/12.22). This unexpected and surprisingly high conversion is beneficial in that there are lower amounts of unreacted C₉ aromatics that need to be recycled back to the reactor for conversion. Though the addition of molybdenum is expected to increase the longevity (activity) of the catalyst, it is unexpected and surprising that the addition of the molybdenum results in such a high conversion of the C₉ aromatics to xylene isomers.

Furthermore, surprisingly and unexpectedly high conversion of the feed is obtainable with catalyst “B” when compared to catalyst “A.” Specifically, the liquid product obtained when using catalyst “A” has a weight ratio of xylene isomers to C₉ aromatics of about 0.37 (i.e., 13.93/37.17). In contrast, the liquid product obtained when passing an identical feed under identical reaction conditions, but using catalyst “B,” has a weight ratio of xylene isomers to C₉ aromatics of about 2.61 (i.e., 31.9/12.22).

Similarly, the data in Table 5 show surprisingly and unexpectedly high conversion of the methylethylbenzene with catalyst “B” when compared to catalyst “A.” Specifically, the liquid product obtained when using catalyst “A” has a weight ratio of methylethylbenzene present in the feed to that present in the product of about 1.71 (i.e., 30.75/18.02). In contrast, the liquid product obtained when passing an identical feed under identical reaction conditions, but using catalyst “B,” has a weight ratio of methylethylbenzene present in the feed to that present in the product of about 33.06 (i.e., 30.75/0.93). This unexpected and surprisingly high conversion is beneficial in that there are lower amounts of unreacted (or produced) methylethylbenzene that need to be recycled back to the reactor for conversion.

Still further, the liquid product obtained when using catalyst “A” has a weight ratio of xylene isomers to ethylbenzene of about 4.64 (i.e., 13.93/3). In contrast, the liquid product obtained when passing an identical feed under identical reaction conditions, but using catalyst “B,” has a weight ratio of xylene isomers to ethylbenzene is about 58 (i.e., 31.9/0.55). This unexpected and surprisingly high weight ratio is beneficial in downstream processing where, as described above, the product stream is to be fractionated into its major constituents, i.e., into aromatics containing 6, 7, 8, and 9 carbons. Typically, further processing of a C₈ aromatics fraction would necessarily involve energy-consuming processing of the ethylbenzene. However, given the substantial absence of ethylbenzene in the liquid reaction product obtained when using catalyst “B,” and the accordingly substantial absence of ethylbenzene in the C₈ aromatics fraction, no such energy-consuming processing is required to rid the fraction of ethylbenzene.

The product obtained with catalyst “B” also has a surprisingly and unexpectedly high amount of xylene isomers to C₁₀ aromatics in comparison to the product obtained using catalyst “A.” Specifically, the liquid product obtained when using catalyst “A” has a weight ratio of xylene isomers to C₁₀ aromatics of about 2.88 (i.e., 13.93/4.83). In contrast, the liquid product obtained when passing an identical feed under identical reaction conditions, but using catalyst “B,” has a weight ratio of xylene isomers to C₁₀ aromatics of about 20.19 (i.e., 31.9/1.58).

Still further, the product obtained with catalyst “B” has a surprisingly and unexpectedly high amount of trimethylbenzene to methylethylbenzene in comparison to the product obtained using catalyst “A.” Specifically, the liquid product obtained when using catalyst “A” has a weight ratio of trimethylbenzene to methylethylbenzene of about 1.05 (i.e., 18.89/18.02). In contrast, the liquid product obtained when passing an identical feed under identical reaction conditions, but using catalyst “B,” has a weight ratio of trimethylbenzene to methylethylbenzene of about 12.14 (i.e., 11.29/0.93).

The product obtained with catalyst “B” has a surprisingly and unexpectedly high amount of benzene to ethylbenzene in comparison to the product obtained using catalyst “A.” Specifically, the liquid product obtained when using catalyst “A” has a weight ratio of benzene to ethylbenzene of about 1.14 (i.e., 3.43/3). In contrast, the liquid product obtained when passing an identical feed under identical reaction conditions, but using catalyst “B,” has a weight ratio of benzene to ethylbenzene of about 20.6 (i.e., 11.3/0.55).

The reported data show that almost 80% of the C₉ aromatics were converted with catalyst “B” (versus only about 39% with catalyst “A”), and about 14% of the toluene in the feed was converted with catalyst “B” (versus only about 7.6% with catalyst “A”). Furthermore, a cursory comparison of the product streams shows that using catalyst “B”: (a) nearly all the methylethylbenzene has been converted; (b) the yields of benzene and xylenes have increased; (c) the concentration of ethylbenzene in the C₈ aromatics is significantly lower; and, (d) the yield of C₁₀ aromatics is drastically reduced. Compared to the reaction of C₉ aromatics alone, there is no net gain in the yield of toluene, while there is an increase in the yield of benzene. Thus, toluene can be co-processed with C₉ aromatics to give increased yields of benzene, if desired, which can be recycled back to the reactor.

### Example 5

This example illustrates the performance capabilities of large-pore, molybdenum-impregnated zeolite catalysts. Specifically, this example illustrates the performance capabilities of a molybdenum-impregnated, mordenite catalyst (Catalyst “B” of Example 1), a molybdenum-impregnated, beta zeolite (Catalyst “C” of Example 1), and a molybdenum-impregnated, USY zeolite (Catalyst “D” of Example 1) to convert a feed comprising about 60 wt % C₉ aromatic (A₉ ) hydrocarbons and about 38 wt % toluene to xylene isomers. Four separate runs were performed with identical feeds. In each run, the catalyst was packed into a ¾-inch tubular, stainless steel, plug-flow reactor and treated with flowing hydrogen for two hours at about 400° C. (752° F.) (unless specified otherwise in the data presented below) and 200 psig (about 1.4 MPa) prior to the introduction of the liquid feed. The feed stream was a mixture of hydrogen and hydrocarbon in a 4:1 molar ratio, and the reaction conditions were set at 400° C. (752° F.) (unless specified otherwise), 200 psig (about 1.4 MPa), and a WHSV of 1.0. Analyses of the liquid feed and product are shown in Table 6, below.

TABLE 6

| | | Catalyst | Catalyst | Catalyst | Catalyst |
|---|---|---|---|---|---|
| | Feed | “B” | “C” | “D” | “D” |
| | Wt % | Pdt. Wt % | Pdt. Wt. % | Pdt. Wt. % | Pdt. Wt. % |
| Process Temp (° F.) | | 750 | 751 | 750 | 771 |
| Light Gas | 0.2 | 10.3 | 11.3 | 9.6 | 8.2 |
| Benzene | 0.1 | 11.3 | 7.8 | 4.8 | 4.5 |
| Toluene | 38.4 | 32.1 | 29.3 | 32.3 | 33.4 |
| Ethylbenzene | 0.0 | 0.5 | 1.9 | 2.9 | 2.6 |
| p-Xylene | 0.1 | 7.7 | 7.5 | 6.4 | 5.8 |
| m-Xylene | 0.3 | 16.9 | 16.3 | 14.1 | 12.7 |
| o-Xylene | 0.2 | 7.3 | 7.1 | 6.2 | 5.7 |
| Propylbenzene | 4.1 | 0.0 | 0.0 | 0.3 | 0.5 |
| Methylethylbenzene | 30.3 | 0.9 | 3.6 | 7.9 | 9.4 |
| Trimethylbenzene | 25.6 | 11.3 | 11.7 | 11.0 | 12.2 |
| A₁₀₊ | 0.7 | 1.6 | 3.5 | 4.5 | 5.0 |

The data set forth in the foregoing table show that, in addition to the molybdenum-impregnated mordenite catalyst (Catalyst “B”), other large-pore molybdenum-impregnated zeolites (Catalysts “C” and “D”) also perform desirably well in converting a C₉ aromatics-comprising feed to xylene isomers. Indeed, these other large-pore molybdenum-impregnated zeolites also produce unexpectedly high ratios of xylene isomers to ethylbenzene, xylene isomers to C₉ aromatics (e.g., methylethylbenzene), xylene isomers to C₁₀ aromatics, trimethylbenzene to methylethylbenzene, benzene to ethylbenzene, in the product of the conversion, and a high conversion of C₉ aromatics and methylethylbenzene.

The foregoing description is given for clearness of understanding only, and no unnecessary limitations should be understood therefrom, as modifications within the scope of the invention may be apparent to those having ordinary skill in the art.

1. A method of making xylene isomers, the method comprising:
(a) contacting a C₉ aromatics-comprising feed with a catalyst under conditions suitable for converting the feed to an intermediate product stream comprising xylene isomers;
(b) separating at least a portion of the xylene isomers from the intermediate product stream; and,
(c) recycling to the feed of step (a) the xylene isomers-lean intermediate product stream obtained in step (b).

2. The method of claim 1, wherein the feed is substantially free of xylene isomers, sulfur, paraffins, and olefins.

3. The method of claim 1, wherein the feed comprises less than about 50 wt. % toluene, based on the total weight of the feed.

4. The method of claim 1, wherein the feed is substantially free of toluene.

5. The method of claim 1, wherein the feed comprises less than about 30 wt. % benzene, based on the total weight of the feed.

6. The method of claim 1, wherein the feed is substantially free of benzene.

7. The method of claim 1, wherein the feed is substantially free of C₁₀₊ aromatics.

8. The method of claim 1, wherein the feed is substantially free of ethylbenzene.

9. The method of claim 1, wherein the contacting step (a) is carried out in the presence of a hydrogen-comprising gas.

10. The method of claim 1, wherein the catalyst comprises a non-sulfided, large-pore zeolite impregnated with a Group VIB metal oxide.

11. The method of claim 10, wherein the zeolite is selected from the group consisting of mordenite, beta-zeolite, and Y-zeolite, and one or more mixtures thereof.

12. The method of claim 11, wherein the zeolite is mordenite.

13. The method of claim 11, wherein the zeolite is beta-zeolite.

14. The method of claim 11, wherein the zeolite is Y-zeolite.

15. The method of claim 10, wherein the Group VIB metal is molybdenum.

16. The method of claim 15, wherein molybdenum comprises about 1.5 wt. % to about 2.5 wt. % of the catalyst, based on the total weight of the catalyst.

17. The method of claim 1, wherein the conditions comprise a temperature of about 200° C. to about 1000° C.

18. The method of claim 1, wherein the conditions comprise a pressure of about 0.5 MPa to about 5 MPa.

19. The method of claim 1, wherein the conditions comprise a WHSV of about 0.1 to about 20.

20. The method of claim 1, wherein the intermediate product stream comprises toluene.

21. The method of claim 20, wherein in step (c), the toluene present in the intermediate product stream is recycled to the feed in step (a).

22. The method of claim 1, wherein the intermediate product stream comprises benzene.

23. The method of claim 22, wherein in step (c), the benzene present in the intermediate product stream is recycled to the feed in step (a).

24. The method of claim 1, wherein the intermediate product stream comprises xylene isomers and ethylbenzene present in a weight ratio of xylene isomers to ethylbenzene of at least about 6 to 1.

25. The method of claim 1, wherein the intermediate product stream comprises xylene isomers and methylethylbenzene present in a weight ratio of xylene isomers to C₉ aromatics of at least about 1 to 1.

26. The method of claim 1, wherein the intermediate product stream comprises xylene isomers and C₁₀ aromatics present in a weight ratio of xylene isomers to C₁₀ aromatics of at least about 3 to 1.

27. The method of claim 1, wherein the intermediate product stream comprises trimethylbenzene and methylethylbenzene present in a weight ratio of trimethylbenzene to methylethylbenzene of at least about 1.5 to 1.

28. The method of claim 1, wherein the intermediate product stream comprises benzene and ethylbenzene present in a weight ratio of benzene to ethylbenzene of at least about 2 to 1.

29. The method of claim 1, wherein the intermediate product stream comprises C₉ aromatics present in a weight ratio of C₉ aromatics in the feed to that in the product of at least about 4 to 1.

30. The method of claim 1, wherein the intermediate product stream comprises methylethylbenzene present in a weight ratio of methylethylbenzene in the feed to that in the product of at least about 2 to 1.

31. The method of claim 1, wherein the intermediate product stream recycled in step (c) comprises less than about 5 wt. % xylene-isomers, based on the total weight of the recycled stream.

32. A method of making xylene isomers, the method comprising contacting a feed comprising C₉ aromatics and less than about 30 wt. % benzene, based on the total weight of the feed, with a non-sulfided, large-pore zeolite impregnated with a Group VIB metal oxide, under conditions suitable for converting the feed to a product stream comprising xylene isomers.

33. The method of claim 32, wherein the feed is substantially free of xylene isomers, sulfur, paraffins, and olefins.

34. The method of claim 32, wherein the feed comprises less than about 50 wt. % toluene.

35. The method of claim 32, wherein the product stream comprises ethylbenzene and xylene isomers in a weight ratio of xylene isomers to ethylbenzene of at least about 6 to 1.

36. A method of converting a C₉ aromatics-comprising feed to a product stream comprising xylene isomers, the method comprising contacting the feed with a catalyst under conditions suitable to yield a weight ratio of xylene isomers to ethylbenzene in the product stream of at least about 6 to 1.

37. The method of claim 36, wherein the feed is substantially free of xylene isomers, sulfur, paraffins, and olefins.

38. The method of claim 36, wherein the feed comprises less than about 50 wt. % toluene.

39. The method of claim 36, wherein the feed comprises less than about 30 wt. % of benzene, based on the total weight of the feed.

40. A method of converting a C₉ aromatics-comprising feed to a product stream comprising xylene isomers, the method comprising contacting the feed with a catalyst under conditions suitable to yield a weight ratio of xylene isomers to methylethylbenzene in the product stream of at least about 1 to 1.

41. The method of claim 40, wherein the feed is substantially free of xylene isomers, sulfur, paraffins, and olefins.

42. The method of claim 40, wherein the feed comprises less than about 50 wt. % toluene.

43. The method of claim 40, wherein the feed comprises less than about 30 wt. % of benzene, based on the total weight of the feed.

44. A method of converting a C₉ aromatics-comprising feed to a product stream comprising xylene isomers, the method comprising contacting the feed with a catalyst under conditions suitable to yield a weight ratio of xylene isomers to C₁₀ aromatics in the product stream of at least about 3 to 1.

45. The method of claim 44, wherein the feed is substantially free of xylene isomers, sulfur, paraffins, and olefins.

46. The method of claim 44, wherein the feed comprises less than about 50 wt. % toluene.

47. The method of claim 44, wherein the feed comprises less than about 30 wt. % of benzene, based on the total weight of the feed.

48. A method of converting a C₉ aromatics-comprising feed to a product stream comprising xylene isomers, the method comprising contacting the feed with a catalyst under conditions suitable to yield a weight ratio of trimethylbenzene to methylethylbenzene in the product stream of at least about 1.5 to 1.

49. The method of claim 48, wherein the feed is substantially free of xylene isomers, sulfur, paraffins, and olefins.

50. The method of claim 48, wherein the feed comprises less than about 50 wt. % toluene.

51. The method of claim 48, wherein the feed comprises less than about 30 wt. % of benzene, based on the total weight of the feed.

52. A method of converting a C₉ aromatics-comprising feed to a product stream comprising xylene isomers, the method comprising contacting the feed with a catalyst under conditions suitable to yield a weight ratio of benzene to ethylbenzene in the product stream of at least about 2 to 1.

53. The method of claim 52, wherein the feed is substantially free of xylene isomers, sulfur, paraffins, and olefins.

54. The method of claim 52, wherein the feed comprises less than about 50 wt. % toluene.

55. The method of claim 52, wherein the feed comprises less than about 30 wt. % of benzene, based on the total weight of the feed.

56. A method of converting a C₉ aromatics-comprising feed to a product stream comprising xylene isomers, the method comprising contacting the feed with a catalyst under conditions suitable to yield a weight ratio of C₉ aromatics present in the feed to that present in the product stream of at least about 4:1.

57. The method of claim 56, wherein the feed is substantially free of xylene isomers, sulfur, paraffins, and olefins.

58. The method of claim 56, wherein the feed comprises less than about 50 wt. % toluene.

59. The method of claim 56, wherein the feed comprises less than about 30 wt. % of benzene, based on the total weight of the feed.

60. A method of converting a C₉ aromatics-comprising feed to a product stream comprising xylene isomers, the method comprising contacting the feed with a catalyst under conditions suitable to yield a weight ratio of methylethylbenzene present in the feed to that present in the product stream of at least about 2:1.

61. The method of claim 60, wherein the feed is substantially free of xylene isomers, sulfur, paraffins, and olefins.

62. The method of claim 60, wherein the feed comprises less than about 50 wt. % toluene.

63. The method of claim 60, wherein the feed comprises less than about 30 wt. % of benzene, based on the total weight of the feed.
